(19) United States
(12) Patent Application Publication
(10) Pub. No.: US 2026/0314156 A1
(43) Pub. Date: Oct. 8, 2026

(54) PHOTOELECTRIC CONVERSION ELEMENT CONTAINING PHOTOELECTRIC CONVERSION ELEMENT MATERIAL

(71) Applicant: NIPPON STEEL Chemical & Material Co., Ltd., Tokyo (JP)

(72) Inventors: Munetomo INOUE, Tokyo (JP); Kentaro HAYASHI, Tokyo (JP)

(73) Assignee: NIPPON STEEL Chemical & Material Co., Ltd., Tokyo (JP)

(21) Appl. No.: 19/132,412

(22) PCT Filed: Dec. 18, 2023
(86) PCT No.: PCT/JP2023/045209

§ 371 Date: May 23, 2025

(30) Foreign Application Priority Data
Dec. 23, 2022 (JP) 2022-206603

(51) Int. Cl. H10K 30/86; H10K 30/40; H10K 85/60; H10K 101/30

(52) U.S. Cl. CPC H10K 30/86; H10K 30/40; H10K 85/622; H10K 85/626; H10K 85/657; H10K 85/6572; H10K 85/6574; H10K 85/6576; H10K 2101/30

(57) ABSTRACT

Provided is a photoelectric conversion device using a material for a photoelectric conversion device that can achieve higher sensitivity and higher resolution. A photoelectric conversion device having one or two or more organic layers between electrodes, in which at least one of the organic layers uses a material for a photoelectric conversion device represented by the following general formula (1). X¹ represents O, S, or N—R¹, R¹ represents an alkyl group having 1 to 20 carbon atoms, an aromatic hydrocarbon group having 6 to 30 carbon atoms, or a heteroaromatic group having 3 to 17 carbon atoms, and Ar¹ to Ar³ represent deuterium, an alkyl group having 1 to 20 carbon atoms, an aromatic hydrocarbon group having 6 to 30 carbon atoms, a heteroaromatic group having 3 to 17 carbon atoms, a group having a tricyclic condensed-ring structure of any of the following formulae (2a) to (2e), or the like.

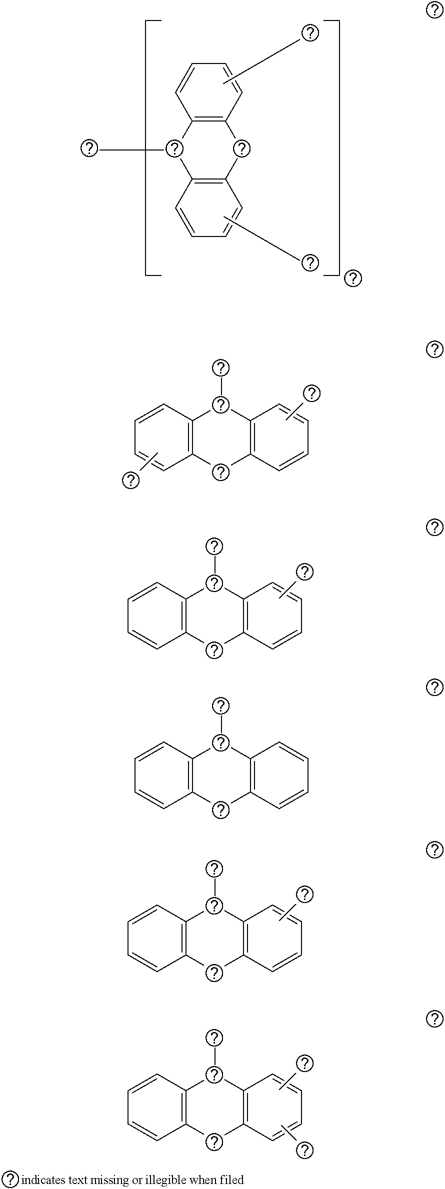

### TECHNICAL FIELD

The present invention relates to a photoelectric conversion element (device) comprising a predetermined material for a photoelectric conversion device, and particularly to a photoelectric conversion device useful for an imaging device.

In recent years, development of an organic electronic device using a thin film formed with an organic semiconductor is in progress. Examples thereof include an electroluminescent device, a solar cell, a transistor device, and a photoelectric conversion device. In particular, development of an organic EL device, which is an electroluminescent device with an organic substance, is most advanced among them. The applications for smartphones, TV and the like are in progress, and development for a purpose of further higher functionality is continuously conducted.

On the photoelectric conversion device, a device using a P-N junction of an inorganic semiconductor, such as silicon, has been conventionally developed and practically used, and made are investigations for high functionalization of a digital camera and a camera for a smartphone and investigation for application for a monitoring camera, a sensor for an automobile, and the like. However, problems for these various uses include improving sensitivity and micronizing a pixel (improving resolution). For the photoelectric conversion device using an inorganic semiconductor, a mainly adopted method for obtaining a color image is disposing color filters corresponding to RGB, which are the three primary colors of light, on a light receiving part of the photoelectric conversion device. This method has problems in terms of utilization efficiency of an incident light and resolution, because the method disposes the RGB color filters on a plane (Non Patent Literature 1 and 2).

As a solution for such problems of the photoelectric conversion device, a photoelectric conversion device using an organic semiconductor instead of the inorganic semiconductor is developed (Non Patent Literature 1 and 2). This utilizes an ability to selectively absorb only light having a specific wavelength region with high sensitivity that the organic semiconductor has, and proposed is stacking photoelectric conversion devices composed of organic semiconductors corresponding to the three primary colors of light to solve the problem of improving the sensitivity and improving the resolution. A device in which a photoelectric conversion device composed of the organic semiconductor and a photoelectric conversion device composed of the inorganic semiconductor are stacked is also proposed (Non Patent Literature 3).

Here, the photoelectric conversion device using the organic semiconductor is a device having a photoelectric conversion layer composed of a thin film of the organic semiconductor between two electrodes, wherein a hole blocking layer and/or an electron blocking layer is disposed between the photoelectric conversion layer and the two electrodes, as necessary. In the photoelectric conversion device, light having a desired wavelength is absorbed in the photoelectric conversion layer to generate an exciton, and then charge separation of the exciton generates a hole and an electron. Thereafter, the hole and the electron move toward each electrode to convert the light into an electric signal. For a purpose of accelerating this process, a method of applying a bias voltage between both the electrodes is commonly used, but one of objects is reducing a leakage current from both the electrodes generated by applying the bias voltage. Accordingly, it can be mentioned that controlling the move of the hole and the electron in the photoelectric conversion device is a key to exhibit characteristic of the photoelectric conversion device.

The organic semiconductor used for each layer of the photoelectric conversion device can be classified into a P-type organic semiconductor and an N-type organic semiconductor. The P-type organic semiconductor is used as a hole transport material, and the N-type organic semiconductor is used as an electron transport material. To control the move of the hole and the electron in the aforementioned photoelectric conversion device, made are various developments of an organic semiconductor having appropriate physical properties such as hole mobility, electron mobility, an energy value of a highest occupied molecular orbital (HOMO), and an energy value of a lowest unoccupied molecular orbital (LUMO). However, the organic semiconductor still has insufficient characteristics, and has not been utilized in commercial practice.

Patent Literature 1 proposes a device using a derivative in which both ends of biscarbazole are each substituted with phenoxazine, for the electron blocking layer disposed between the photoelectric conversion layer and the electrode.

Patent Literatures 2 and 3 propose a device using a naphthalene derivative to which a carbazole backbone is linked as a tricyclic condensed-ring structure, for the electron blocking layer disposed between the photoelectric conversion layer and the electrode.

Patent Literature 4 proposes a device using a carbazole derivative to which a carbazole backbone is linked as a tricyclic condensed-ring structure, for the electron blocking layer disposed between the photoelectric conversion layer and the electrode.

Patent Literature 5 proposes a device using a spirofluorene derivative to which an acridine backbone is linked as a tricyclic condensed-ring structure, for the electron blocking layer disposed between the photoelectric conversion layer and the electrode.

### CITATION LIST

### Patent Literature

### Patent Literature 1

JP 2011-228614 (A)

### Patent Literature 2

JP 2019-005919 (A)

### Patent Literature 3

WO2018/235780

### Patent Literature 4

JP 2015-153910 (A)

### Patent Literature 5

JP 2011-82507 (A)

### Non Patent Literature

### Non Patent Literature 1

NHK Science & Technology Research Laboratories R&D No. 132, pp. 4-11 (2012.3)

### Non Patent Literature 2

NHK Science & Technology Research Laboratories R&D No. 174, pp. 4-17 (2019.3)

Non Patent Literature 3
2019 IEEE International Electron Devices Meeting (IEDM), pp. 16.6.1-16.6.4 (2019)

### SUMMARY OF INVENTION

### Technical Problem

For example, in the use of the photoelectric conversion device for imaging for highly functionalizing a digital camera and a camera for a smartphone and for application for a monitoring camera, a sensor for an automobile, and the like, challenges are further higher sensitivity and higher resolution. In view of such a circumstance, an object of the present invention is to provide a material that achieves higher sensitivity and higher resolution of the photoelectric conversion device, and a photoelectric conversion device using the same,

### Solution to Problem

The present inventors have made intensive investigation, and consequently found that using a compound having a specific tricyclic condensed-ring structure efficiently proceeds a process of generating a hole and an electron by charge separation of an exciton in a photoelectric conversion layer, and a process of moving of the hole and the electron in the photoelectric conversion device. This finding has led to the completion of the present invention.

That is, the present invention relates to a photoelectric conversion device comprising one or two or more organic layers between two electrodes, wherein at least one of the organic layers contains a material for a photoelectric conversion device represented by the following general formula (1).

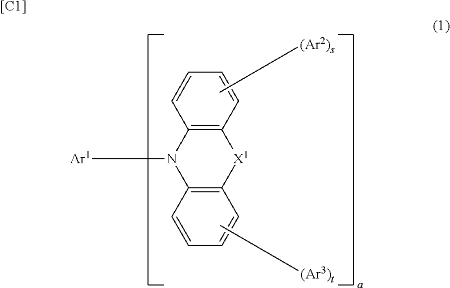

In the general formula (1), X¹ is each independently represented by O, S, or N—R¹, and X¹ is each independently preferably represented by O or S, more preferably represented by O.

R¹ each independently represents an alkyl group having 1 to 20 carbon atoms, a substituted or unsubstituted aromatic hydrocarbon group having 6 to 30 carbon atoms, or a substituted or unsubstituted heteroaromatic group having 3 to 17 carbon atoms.

Ar¹ to Ar³ each independently represent deuterium, an alkyl group having 1 to 20 carbon atoms, a substituted or unsubstituted aromatic hydrocarbon group having 6 to 30 carbon atoms, a substituted or unsubstituted heteroaromatic group having 3 to 17 carbon atoms, or a group having a tricyclic condensed-ring structure represented by any of the following formulae (2a) to (2e), or a substituted or unsubstituted linked aromatic group in which two to five of any of a substituted or unsubstituted aromatic hydrocarbon group having 6 to 30 carbon atoms, a substituted or unsubstituted heteroaromatic group having 3 to 17 carbon atoms, and the following formulae (2a) to (2e) are linked. Note that Ar¹ to Ar³ are each independently represented by the formula (2c) or (2d) when the tricyclic condensed-ring structure is contained at the terminal, or Ar¹ to Ar³ are each independently represented by the formula (2a), (2b), or (2e) when the tricyclic condensed-ring structure is not contained at the terminal and there are two bonding sites. Ar¹ to Ar³ each independently preferably represent deuterium, a substituted or unsubstituted aromatic hydrocarbon group having 6 to 18 carbon atoms, a substituted or unsubstituted heteroaromatic group having 3 to 12 carbon atoms, or a group having a tricyclic condensed-ring structure represented by any of the following formulae (2a) to (2e), or a substituted or unsubstituted linked aromatic group in which two to three of any of a substituted or unsubstituted aromatic hydrocarbon group having 6 to 30 carbon atoms, a substituted or unsubstituted heteroaromatic group having 3 to 17 carbon atoms, and the following formulae (2a) to (2e) are linked.

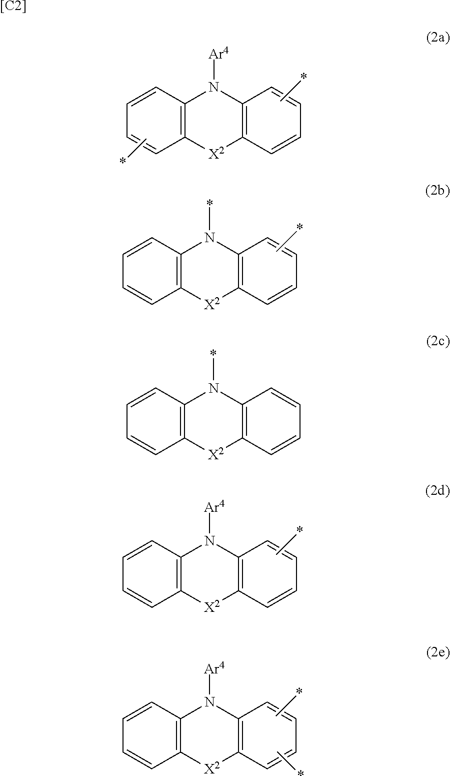

wherein “*” represents a point of bonding.

Ar⁴ independently represents hydrogen, deuterium, an alkyl group having 1 to 20 carbon atoms, a substituted or unsubstituted aromatic hydrocarbon group having 6 to 30 carbon atoms, or a substituted or unsubstituted linked aromatic group in which two to five of these aromatic groups are linked.

X² is each independently represented by a single bond, O, S, or N—R², and X² is preferably represented by O or S.

R² each independently represents an alkyl group having 1 to 20 carbon atoms, a substituted or unsubstituted aromatic hydrocarbon group having 6 to 30 carbon atoms, or a substituted or unsubstituted heteroaromatic group having 3 to 17 carbon atoms.

“s” and “t” independently represent the number of substitution, “s” and “t” independently represent an integer of 0 to 2, and “s” and “t” are independently preferably represented by 0 to 1.

“a” represents the number of substitution and represents an integer of 1 or 2.

Herein, when “a” is 1, preferably, at least one of Ar¹ to Ar³ contains the group having the tricyclic condensed-ring structure represented by any of the formulae (2a) to (2e), and at least two thereof contain the group having the tricyclic condensed-ring structure represented by any of the formulae (2a) to (2e). In this case, when any of Ar¹ to Ar³ is a linked aromatic group, the linked aromatic group may contain two of such groups having the tricyclic condensed-ring structure represented by any of the formulae (2a) to (2e).

Preferably, “a” is 1 and at least one of Ar¹ to Ar³ contains the group having the tricyclic condensed-ring structure represented by the formula (2c), or more preferably, at least two of Ar¹ to Ar³ contain the group having the tricyclic condensed-ring structure represented by the formula (2c). In the above case, when Ar² or Ar³ contains the group having the tricyclic condensed-ring structure represented by any of the formulae (2a) to (2e), “s” and “t” representing the number of substitution are not 0, but 1 or 2.

In the general formula (1), Ar¹ preferably contains any ring structure represented by the following general formulae (3a) to (3m), and Art is more preferably any ring structure represented by the following general formulae (3a) to (3m).

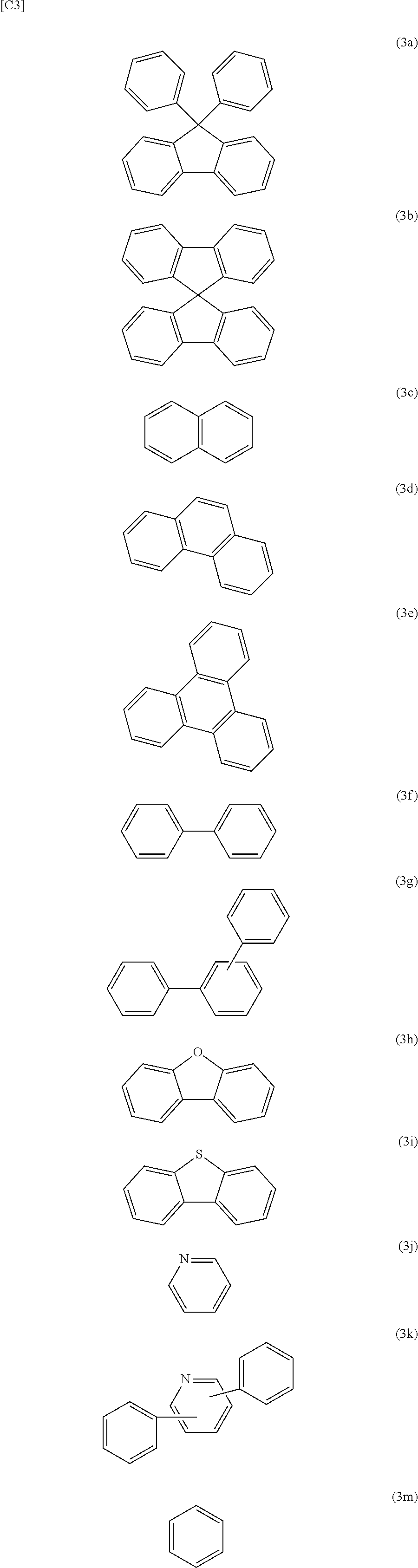

When Ar¹ is represented by the general formula (3b), bonding is preferably made at a bonding position as shown in the following general formula (3b-1) or (3b-2), and examples include the following (P38) or (P39):

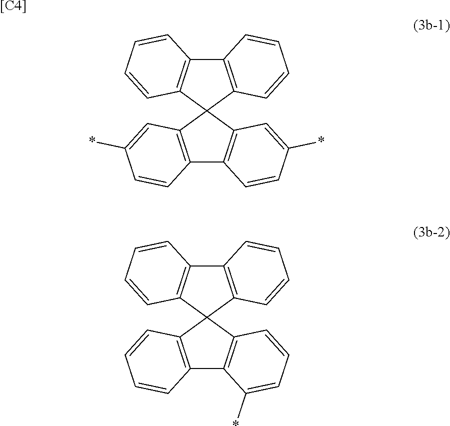

wherein “*” represents a point of bonding to N in the formula (1).

Ar¹ also preferably contains at least one substituted or unsubstituted heteroaromatic group having 3 to 17 carbon atoms and containing an oxygen or sulfur atom.

The material for a photoelectric conversion device represented by the general formula (1) preferably satisfies any one of requirements that: an energy level of highest occupied molecular orbital (HOMO) obtained by structural optimization calculation with density functional calculation B3LYP/6-31G (d) is −4.0 eV or lower; an energy level of lowest unoccupied molecular orbital (LUMO) obtained by the structural optimization calculation is −2.5 eV or higher; the material has a hole mobility of 1×10⁻⁶ cm²/Vs or more; or the material is amorphous. An energy level of HOMO is preferably −4.5 eV or less and −5.2 eV or more.

The material for a photoelectric conversion device represented by the general formula (1) can be used as the hole transport material in the photoelectric conversion device for imaging.

The present invention is a photoelectric conversion device for imaging, comprising a photoelectric conversion layer and an electron blocking layer between two electrodes, wherein at least one layer of the photoelectric conversion layer and the electron blocking layer contains the material for a photoelectric conversion device represented by the general formula (1).

In the present invention, the above material for a photoelectric conversion device represented by the general formula (1) is preferably contained in an electron blocking layer or a photoelectric conversion layer of the photoelectric conversion device, and in this case, preferably contained as a hole transport material. The above material for a photoelectric conversion device is preferably used in the electron blocking layer. When the material for a photoelectric conversion device is contained in the electron blocking layer, the photoelectric conversion layer preferably contains an electron transport material, in particular, preferably contains a fullerene derivative as an electron transport material. In this case, the photoelectric conversion layer preferably further contains a material having a backbone containing at least two thiophene rings, as a hole transport material.

In the present invention, when the material for a photoelectric conversion device represented by the general formula (1) is used in the electron blocking layer of the photoelectric conversion device, the absolute value (|h1−e1|) of a difference in energy level between the HOMO energy level (h1) of the hole transport material used in the photoelectric conversion layer adjacent to the electron blocking layer and the HOMO energy level (e1) of the material for a photoelectric conversion device represented by the general formula (1), contained in the electron blocking layer, is preferably 1.0 eV or less, more preferably 0.8 eV or less.

When the material for a photoelectric conversion device represented by the general formula (1) is used in the photoelectric conversion layer of the photoelectric conversion device, the absolute value (|h2−e21) of a difference in energy level between the HOMO energy level (h2) of the hole transport material used in the electron blocking layer adjacent to the photoelectric conversion layer and the HOMO energy level (e2) of the material for a photoelectric conversion device represented by the general formula (1), contained in the photoelectric conversion layer, is preferably 1.0 eV or less, more preferably 0.8 eV or less.

When the photoelectric conversion layer contains a plurality of materials, the hole transport material contained in the photoelectric conversion layer indicates a material having a volume fraction of at least 5% or more and exhibiting a largest HOMO energy level among the materials contained in the photoelectric conversion layer. Similarly, when the electron blocking layer contains a plurality of materials, the hole transport material contained in the electron blocking layer indicates a material having a volume fraction of at least 5% or more and exhibiting a largest HOMO energy level among the materials contained in the electron blocking layer.

### Advantageous Effect of Invention

The material for a photoelectric conversion device in the present invention can achieve appropriate move of the hole and the electron in the photoelectric conversion device, and consequently enables to reduce a leakage current generated by applying a bias voltage during the conversion of light into electric energy in a photoelectric conversion device. As a result, a photoelectric conversion device that achieves a low dark current value and a high contrast ratio can be obtained, and the material is suitably useful particularly as a material for a photoelectric conversion device for a photoelectric-converting film-stacked imaging device.

### BRIEF DESCRIPTION OF DRAWING

FIG. 1 is a sectional schematic view illustrating a structure example of a photoelectric conversion device for imaging.

### DESCRIPTION OF EMBODIMENTS

The photoelectric conversion device for imaging of the present invention has at least one organic layer (namely, one or two or more organic layers) between two electrodes. In the present invention, such at least one organic layer contains the material for a photoelectric conversion device represented by the general formula (1). Suitably, in a photoelectric conversion device for imaging, comprising a photoelectric conversion layer and an electron blocking layer between two electrodes, at least one layer of the photoelectric conversion layer and the electron blocking layer contains the material for a photoelectric conversion device represented by the general formula (1).

Hereinafter, the material for a photoelectric conversion device represented by the general formula (1) may be referred to as a material for a photoelectric conversion device of the present invention, or may be simply referred to as a material for a photoelectric conversion device, a material of the present invention, or the compound represented by the general formula (1).

The compound represented by the general formula (1) will be described below.

X¹ is each independently represented by O, S, or N—R¹, and X¹ is each independently preferably represented by O or S, more preferably represented by O.

R¹ each independently represents an alkyl group having 1 to 20 carbon atoms, a substituted or unsubstituted aromatic hydrocarbon group having 6 to 30 carbon atoms, or a substituted or unsubstituted heteroaromatic group having 3 to 17 carbon atoms.

When R¹ is an unsubstituted alkyl group having 1 to 20 carbon atoms, the alkyl group may be any of a linear, branched, or cyclic alkyl group, and is preferably a linear, branched, or cyclic alkyl group having 1 to 10 carbon atoms. Specific examples thereof include linear saturated hydrocarbon groups, such as a methyl group, an ethyl group, a n-propyl group, a n-butyl group, a n-pentyl group, a n-hexyl group, a n-octyl group, a n-dodecyl group, a n-tetradecyl group, and a n-octadecyl group, branched saturated hydrocarbon groups, such as an isopropyl group, an isobutyl group, a tert-butyl group, a neopentyl group, a 2-ethylhexyl group, and a 2-hexyloctyl group, and saturated alicyclic hydrocarbon groups, such as a cyclopentyl group, a cyclohexyl group, a cyclooctyl group, a 4-butylcyclohexyl group, and a 4-dodecylcyclohexyl group. The alkyl group having 1 to 20 carbon atoms may be any of a linear, branched, or cyclic alkyl group.

When R¹ represents an unsubstituted aromatic hydrocarbon compound having 6 to 30 carbon atoms, the number of carbon atoms is preferably 6 to 18. Examples of the unsubstituted aromatic hydrocarbon compound having 6 to 30 carbon atoms include a group generated from: monocyclic aromatic hydrocarbons, such as benzene; bicyclic aromatic hydrocarbons, such as naphthalene; tricyclic aromatic hydrocarbons, such as indacene, biphenylene, phenalene, anthracene, phenanthrene, and fluorene; tetracyclic aromatic hydrocarbons, such as fluoranthene, acephenanthrylene, aceanthrylene, triphenylene, pyrene, chrysene, tetraphene, tetracene, and pleiadene; pentacyclic aromatic hydrocarbons, such as picene, perylene, pentaphene, pentacene, tetraphenylene, and naphthoanthracene; and the like. The unsubstituted aromatic hydrocarbon compound is a group generated from preferably benzene, naphthalene, anthracene, phenanthrene, triphenylene, or pyrene, and more preferably benzene, naphthalene, anthracene, phenanthrene, pyrene, or triphenylene.

When R¹ represents an unsubstituted heteroaromatic group having 3 to 17 carbon atoms, the number or carbon atoms is preferably 3 to 12, and the number or carbon atoms is more preferably 6 to 12. Examples of the heteroaromatic group having 3 to 17 carbon atoms include a group generated from: nitrogen-containing aromatic compounds having a pyrrole ring, such as pyrrole, pyrrolopyrrole, indole, isoindole, pyrroloisoindole, and carboline; thiophene, benzothiophene, dibenzothiophene, furan, benzofuran, dibenzofuran, carbazole, indolocarbazole, pyridine, pyrimidine, quinoline, isoquinoline, quinazoline, quinoxaline, or the like. The heteroaromatic group is preferably a group generated from thiophene, benzothiophene, dibenzothiophene, furan, benzofuran, dibenzofuran, or carbazole, more preferably dibenzothienyl, dibenzofuranyl, or carbazolyl.

Ar¹ to Ar³ each independently represent deuterium, an alkyl group having 1 to 20 carbon atoms, a substituted or unsubstituted aromatic hydrocarbon group having 6 to 30 carbon atoms, a substituted or unsubstituted heteroaromatic group having 3 to 17 carbon atoms, or the group having the tricyclic condensed-ring structure represented by any of the formulae (2a) to (2e), or a substituted or unsubstituted linked aromatic group in which two to five of any of a substituted or unsubstituted aromatic hydrocarbon group having 6 to 30 carbon atoms, a substituted or unsubstituted heteroaromatic group having 3 to 17 carbon atoms, and the formulae (2a) to (2e) are linked. Note that Ar¹ to Ar³ are each independently represented by the formula (2c) or (2d) when the tricyclic condensed-ring structure is contained at the terminal, or Ar¹ to Ar³ are each independently represented by the formula (2a), (2b), or (2e) when the tricyclic condensed-ring structure is not contained at the terminal and there are two bonding sites. Ar¹ to Ar³ each independently preferably represent deuterium, a substituted or unsubstituted aromatic hydrocarbon group having 6 to 18 carbon atoms, a substituted or unsubstituted heteroaromatic group having 3 to 12 carbon atoms, or a group having a tricyclic condensed-ring structure represented by any of the following formulae (2a) to (2e), or a substituted or unsubstituted linked aromatic group in which two to three of any of an aromatic hydrocarbon group having 6 to 18 carbon atoms, a heteroaromatic group having 3 to 17 carbon atoms, and the following formulae (2a) to (2e) are linked.

When Ar¹ to Ar³ represent an alkyl groups having 1 to 20 carbon atoms, the number of carbon atoms is preferably 1 to 10, Examples of the case of the alkyl group having 1 to 20 carbon atoms are same as the case described in R¹.

When Ar¹ to Ar³ represent an unsubstituted aromatic hydrocarbon group having 6 to 30 carbon atoms, the number of carbon atoms is preferably 6 to 18. Examples of the aromatic hydrocarbon group having 6 to 30 carbon atoms are same as the case described in R¹.

When Ar¹ to Ar³ represent an unsubstituted heteroaromatic group having 3 to 17 carbon atoms, the number of carbon atoms is preferably 6 to 12. Examples of the heteroaromatic group having 3 to 17 carbon atoms include, in addition to the case described with respect to R¹, the groups having the tricyclic condensed-ring structures represented by the formulae (2a) to (2e).

When Ar¹ to Ar³ represent an unsubstituted linked aromatic group in which two to five of an unsubstituted aromatic hydrocarbon group having 6 to 30 carbon atoms, an unsubstituted heteroaromatic group having 3 to 17 carbon atoms, and the formulae (2a) to (2e) are linked, the number of linkage is preferably 2 to 3 and the number of linkage is more preferably 2. When Ar¹ to Ar³ represent the linked aromatic group, the groups described in Ar¹ to Ar³, and the formula (1) may be bonded to any of the aromatic hydrocarbon group, the heteroaromatic group, or the formulae (2a) to (2e) contained in the linked aromatic group, and may be bonded at both ends or may be bonded in a branched form.

Ar⁴ independently represents deuterium, an alkyl group having 1 to 20 carbon atoms, a substituted or unsubstituted aromatic hydrocarbon group having 6 to 30 carbon atoms, or a substituted or unsubstituted linked aromatic group in which two to five of these aromatic groups are linked. When Ar⁴ represents an alkyl group having 1 to 20 carbon atoms, the number of carbon atoms is preferably 1 to 10. Examples of the alkyl group having 1 to 20 carbon atoms are same as the case described in R¹. When Ar⁴ represents an unsubstituted aromatic hydrocarbon group having 6 to 30 carbon atoms, the number of carbon atoms is preferably 6 to 18. Examples of the aromatic hydrocarbon group having 6 to 30 carbon atoms are same as the case described in R¹.

X² is each independently represented by a single bond, O, S, or N—R², and X² is preferably represented by O or S.

R² each independently represents an alkyl group having 1 to 20 carbon atoms, a substituted or unsubstituted aromatic hydrocarbon group having 6 to 30 carbon atoms, or a substituted or unsubstituted heteroaromatic group having 3 to 17 carbon atoms. When R² represents an unsubstituted alkyl group having 1 to 20 carbon atoms, the number of carbon atoms is preferably 1 to 10. Examples of the alkyl group having 1 to 20 carbon atoms are same as the case described in R¹. When Ar⁴ represents an unsubstituted aromatic hydrocarbon group having 6 to 30 carbon atoms, the number of carbon atoms is preferably 6 to 18. Examples of the aromatic hydrocarbon group having 6 to 30 carbon atoms are same as the case described in R¹. When Ar⁴ represents an unsubstituted heteroaromatic group having 3 to 17 carbon atoms, the number of carbon atoms is preferably 3 to 12. Examples of the heteroaromatic group having 3 to 17 carbon atoms are same as the case described in R¹.

“s” and “t” independently represent the number of substitution, “s” and “t” independently represent an integer of 0 to 2, and “s” and “t” are independently represented by 0 to 1.

“a” represents the number of substitution and represents an integer of 1 or 2. Herein, when “a” is 1, preferably, at least one of Ar¹ to Ar³ contains the group having the tricyclic condensed-ring structure represented by any of the formulae (2a) to (2e) and at least two thereof contain the group having the tricyclic condensed-ring structure represented by any of the formulae (2a) to (2e). When any of Ar¹ to Ar³ represents a linked aromatic group, the linked aromatic group preferably contains two of such groups having the tricyclic condensed-ring structure represented by any of the formulae (2a) to (2e).

Preferably, “a” is 1 and at least one of Ar¹ to Ar³ contains the group having the tricyclic condensed-ring structure represented by the formula (2c), or more preferably at least two of Ar¹ to Ar³ contain the group having the tricyclic condensed-ring structure represented by the formula (2c).

Ar¹ preferably contains any ring structure represented by the formulae (3a) to (3m), and Ar¹ is more preferably any ring structure represented by the general formulae (3a) to (3m).

Among the ring structures represented by the formulae (3a) to (3m), those represented by the formulae (3b), (3e), (3f), (3g), (3h), (31), and (3m) are preferable, and those represented by the formulae (3b), (3e), (3f), (3h), and (3m) are more preferable.

Ar¹ also preferably contains at least one substituted or unsubstituted heteroaromatic group having 3 to 17 carbon atoms and containing an oxygen or sulfur atom. Examples include a heteroaromatic group containing a furan ring or a thiophene ring and having a ring in which such furan ring or thiophene ring and an aromatic hydrocarbon ring are condensed, and an aromatic hydrocarbon ring to be condensed may be present singly or plurally, but not limited thereto.

The unsubstituted linked aromatic group herein refers to an aromatic group in which two or more aromatic groups selected from the above aromatic hydrocarbon group, the aromatic group in the heteroaromatic group, and the groups having the tricyclic condensed-ring structures represented by the formulae (2a) to (2e) are bonded and linked via a single bond. Such a linked aromatic group may be linear or branched. The linking position at which benzene rings are linked may be any of the ortho-, meta-, and para-positions, and linking at the para-position or the meta-position is preferable. The aromatic group may be the aromatic hydrocarbon group, the heteroaromatic group, or any of the groups having the tricyclic condensed-ring structures represented by the formulae (2a) to (2e), and a plurality of such aromatic groups may be the same or different.

Examples of the substituent with which the aromatic hydrocarbon group or the like is optionally substituted, such as a substituted or unsubstituted aromatic hydrocarbon group having 6 to 30 carbon atoms, herein include deuterium, a cyano group, or an alkyl group having 1 to 20 carbon atoms.

In particular, when the substituent is an alkyl group having 1 to 20 carbon atoms, the alkyl group may be any of a linear, branched, or cyclic alkyl group, and is preferably a linear, branched, or cyclic alkyl group having 1 to 10 carbon atoms. Specific examples thereof include: linear saturated hydrocarbon groups, such as a methyl group, an ethyl group, a n-propyl group, a n-butyl group, a n-pentyl group, a n-hexyl group, a n-octyl group, a n-dodecyl group, a n-tetradecyl group, and a n-octadecyl group; branched saturated hydrocarbon groups, such as an isopropyl group, an isobutyl group, a tert-butyl group, a neopentyl group, a 2-ethylhexyl group, and a 2-hexyloctyl group; and saturated alicyclic hydrocarbon groups, such as a cyclopentyl group, a cyclohexyl group, a cyclooctyl group, a 4-butylcyclohexyl group, and a 4-dodecylcyclohexyl group.

In the present invention, the substituent is bonded to a carbon atom or a heteroatom that constitute the aromatic ring.

Preferable specific examples of the material for a photoelectric conversion device represented by the general formula (1) of the present invention will be described below, but the material is not limited thereto. The number in parentheses represents a compound number.

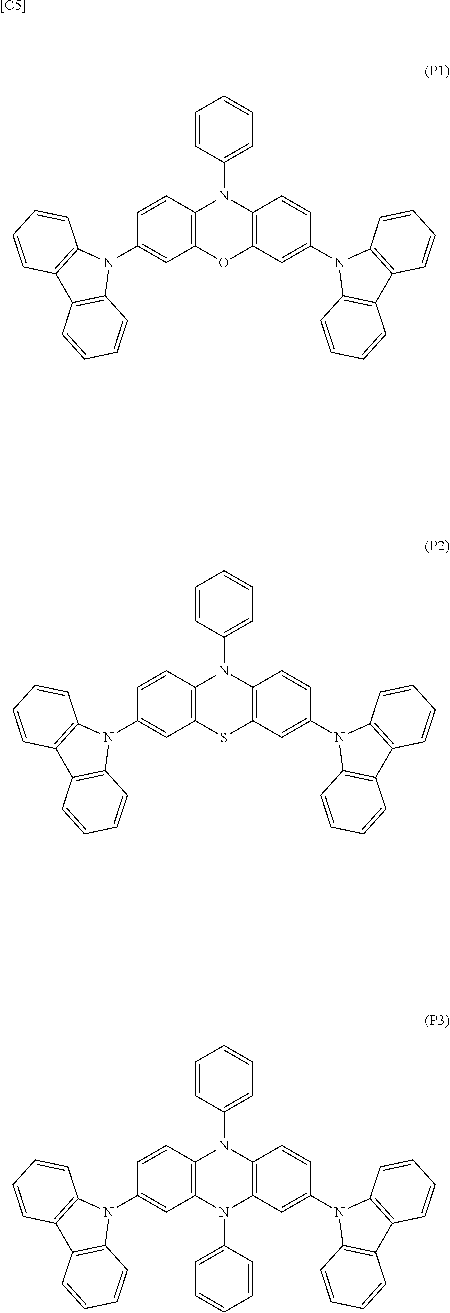

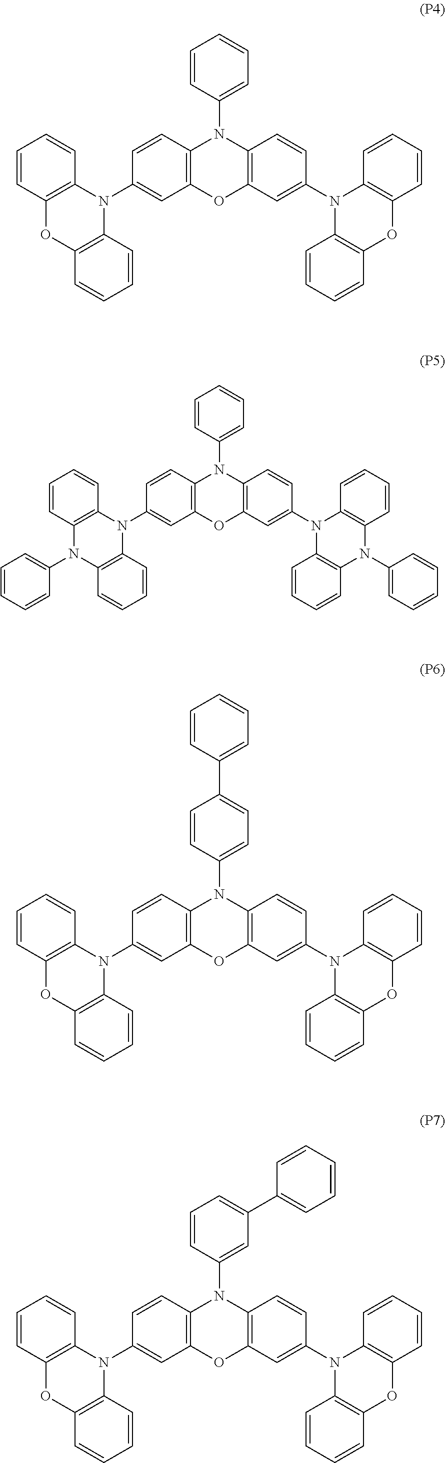

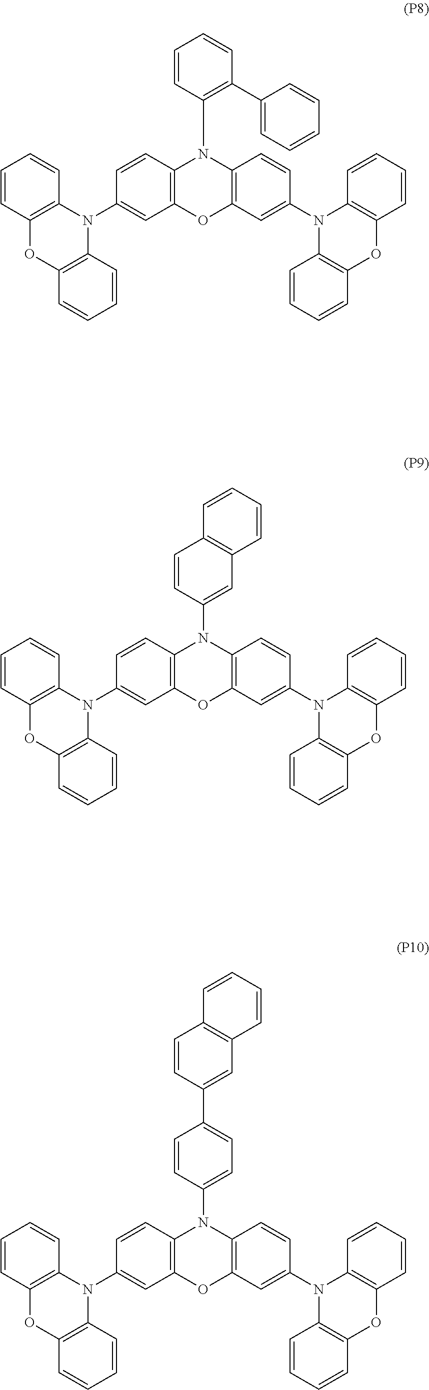

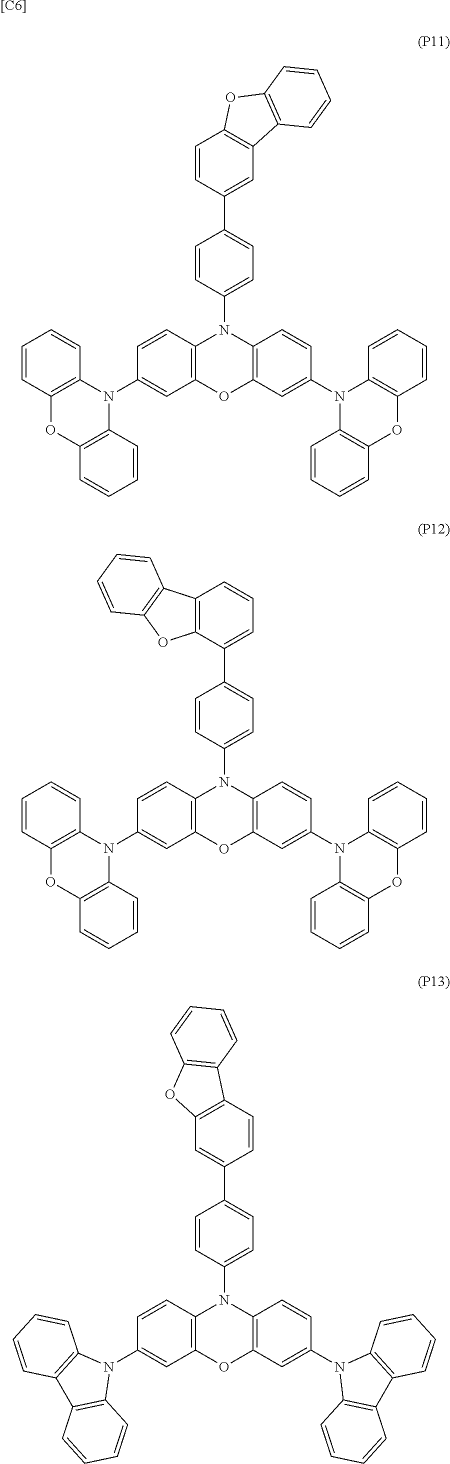

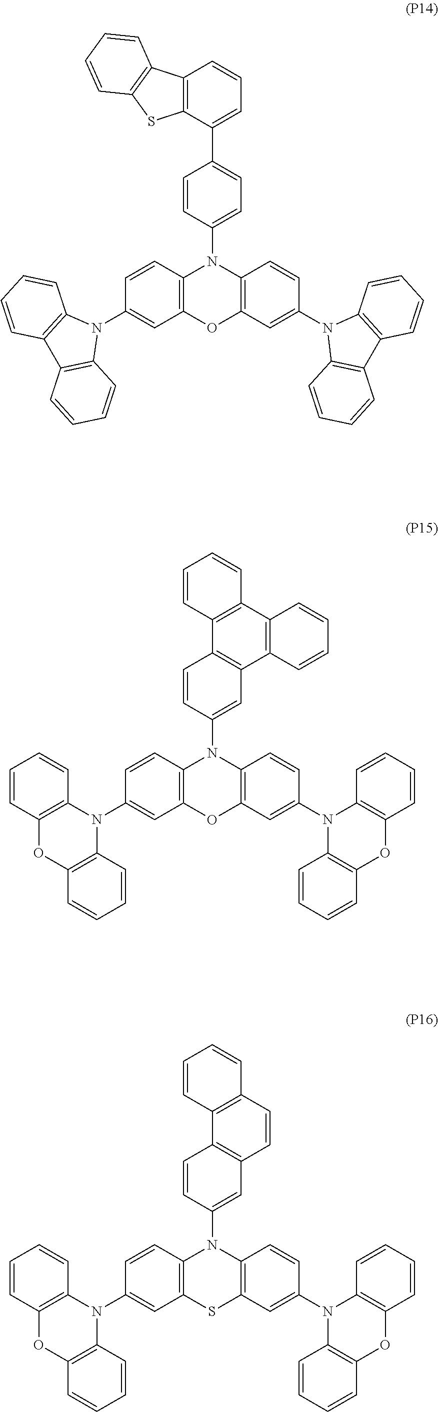

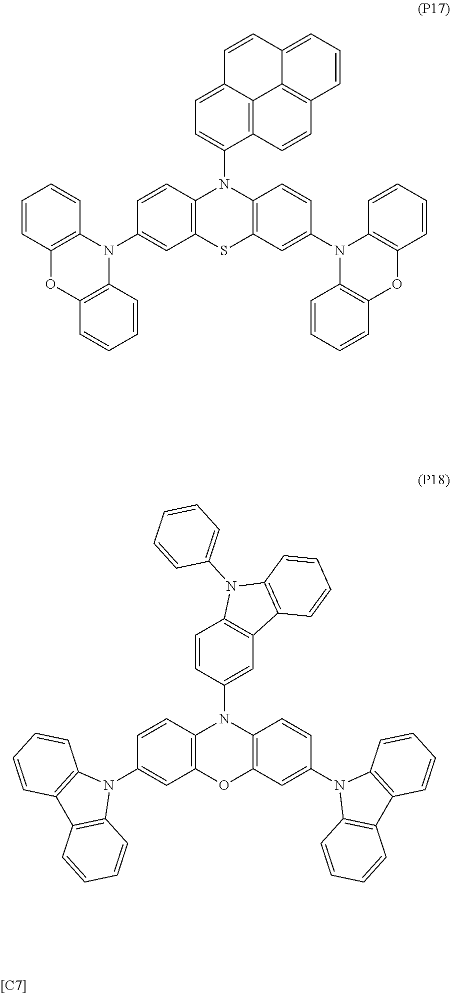

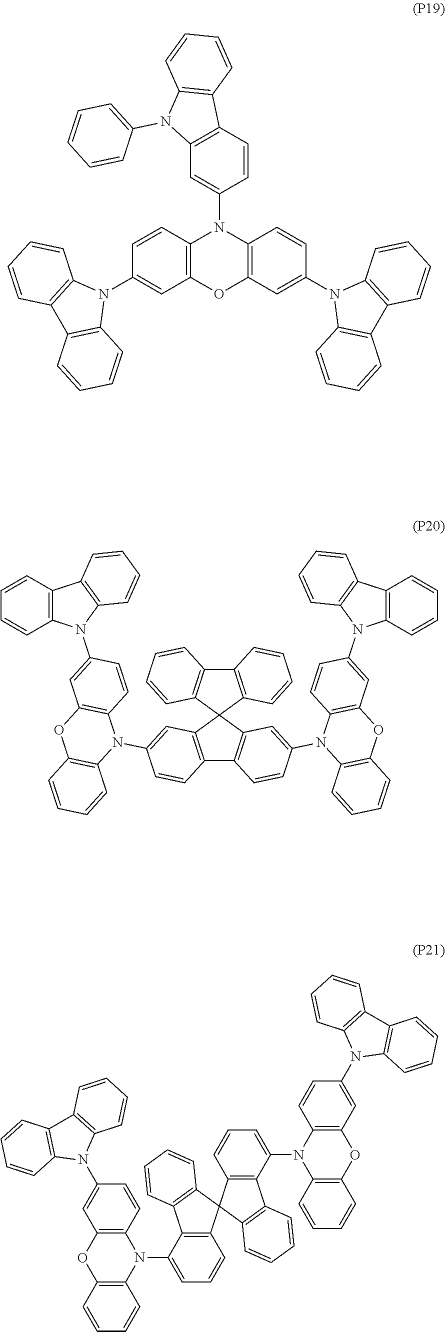

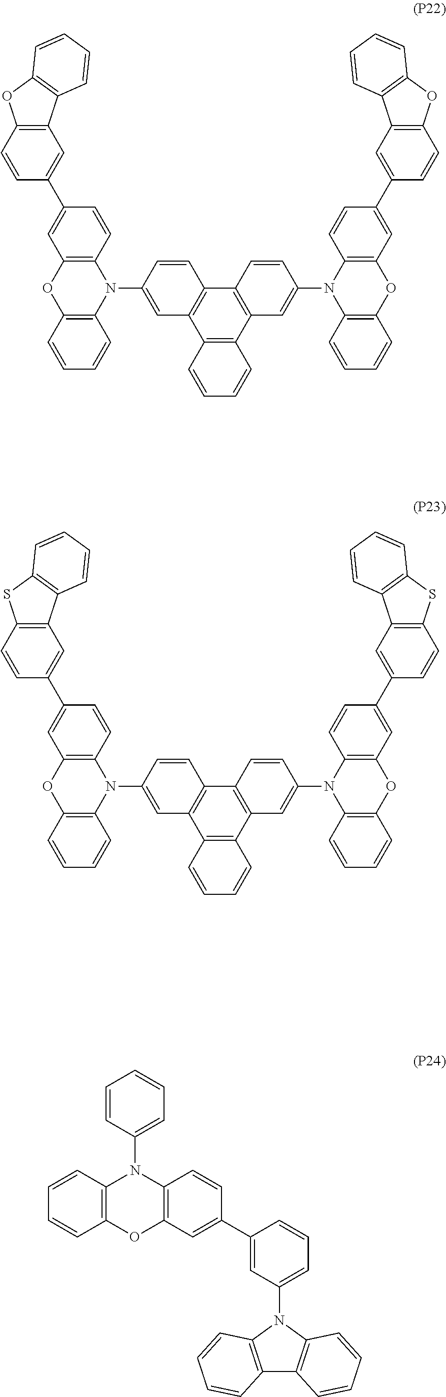

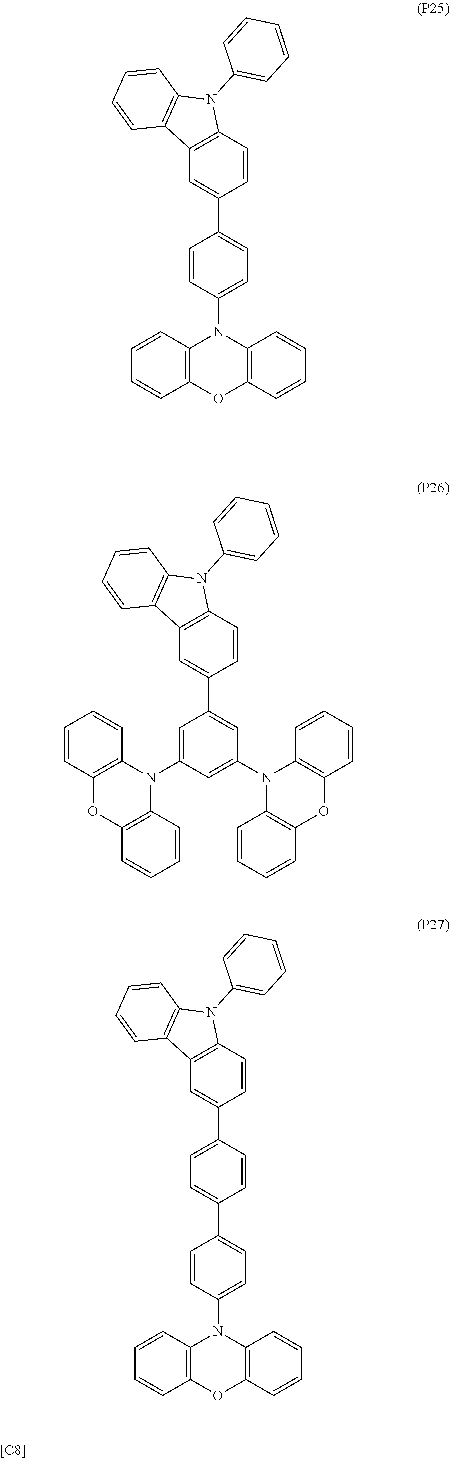

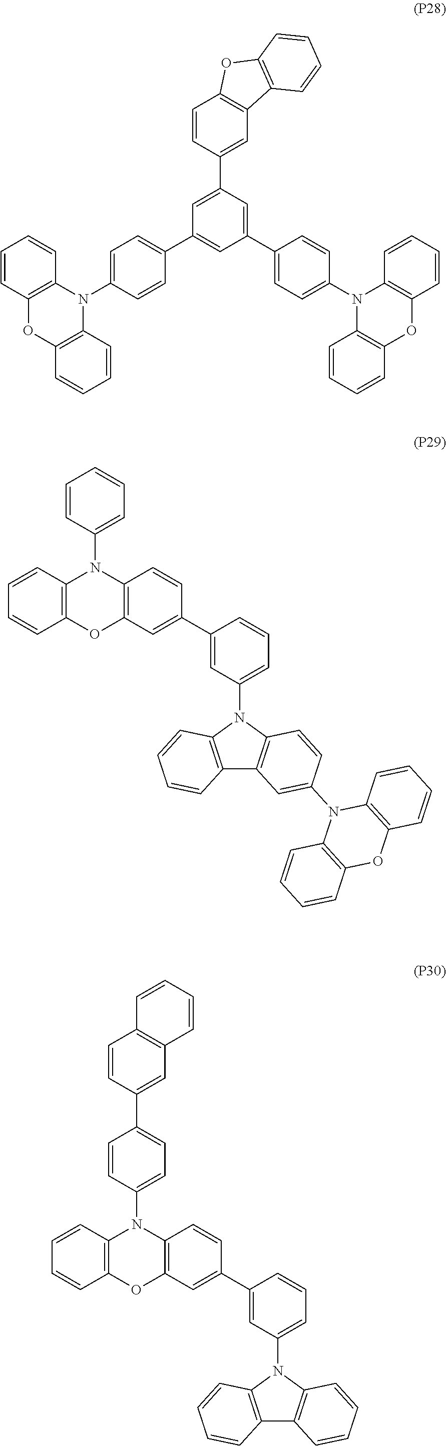

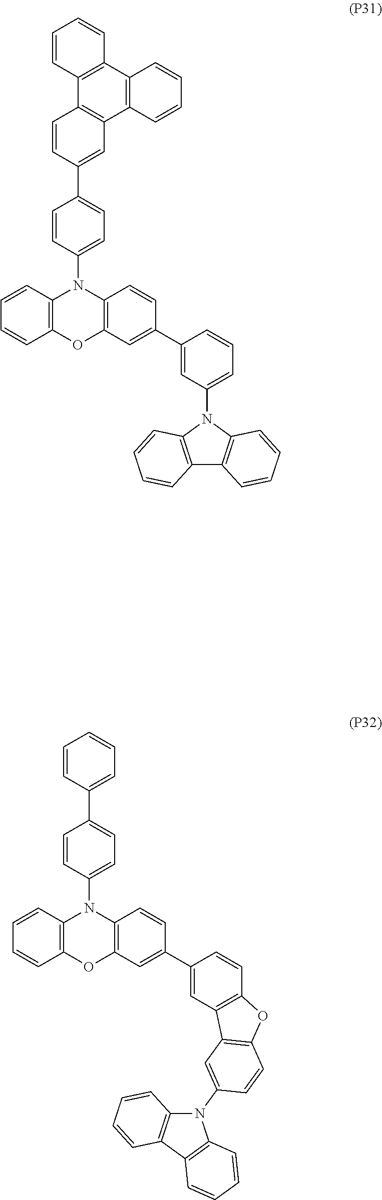

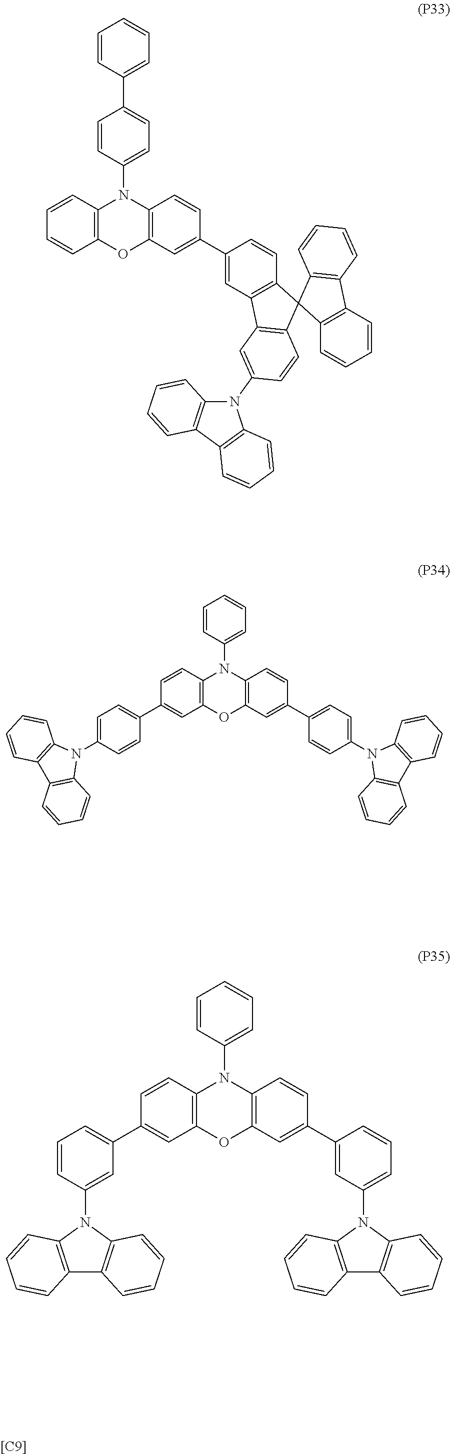

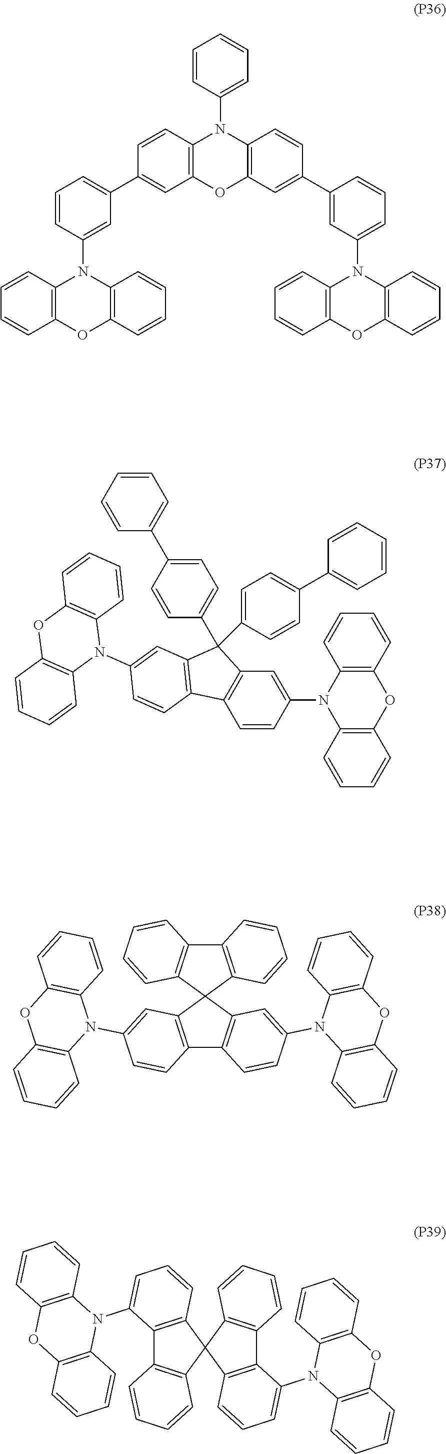

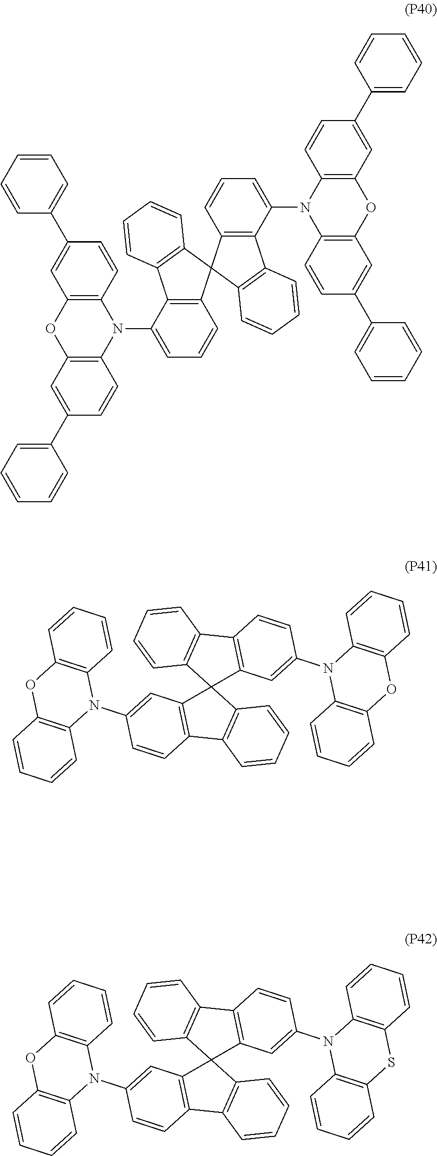

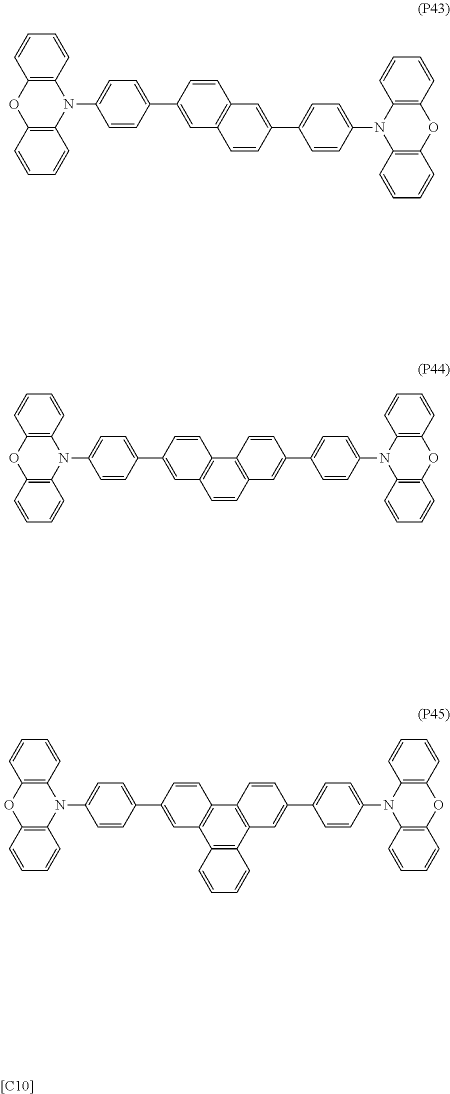

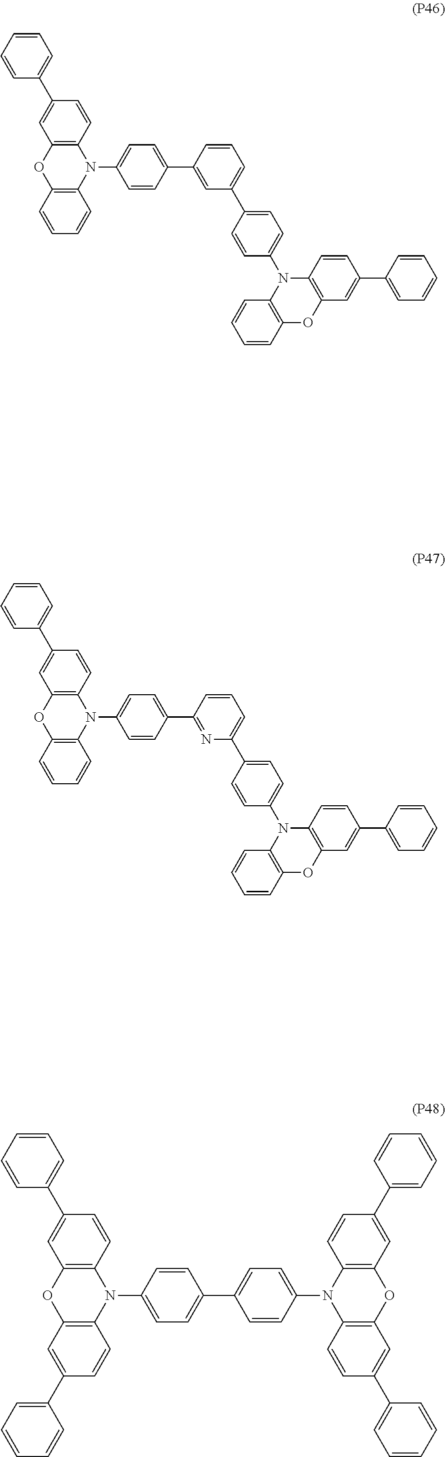

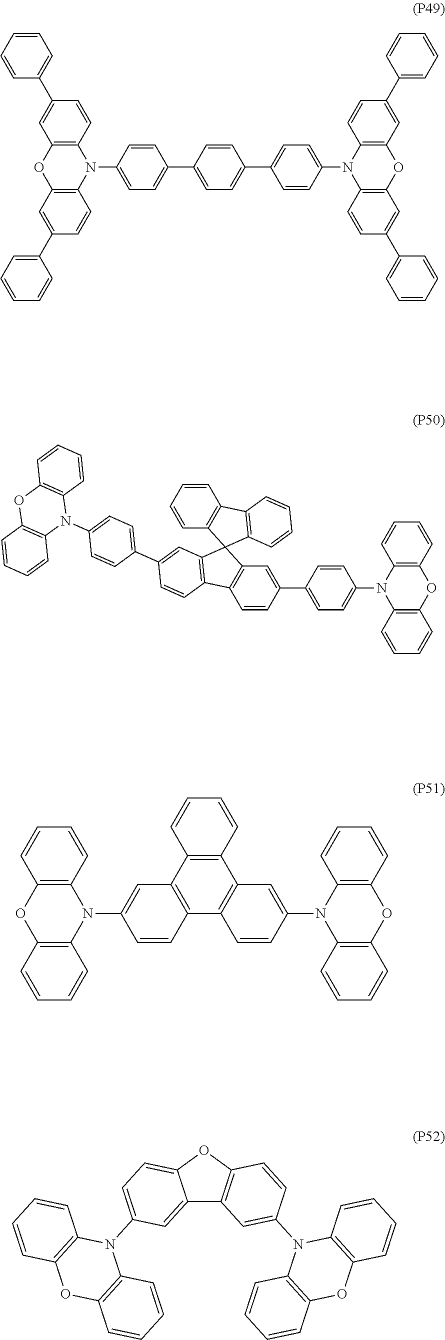

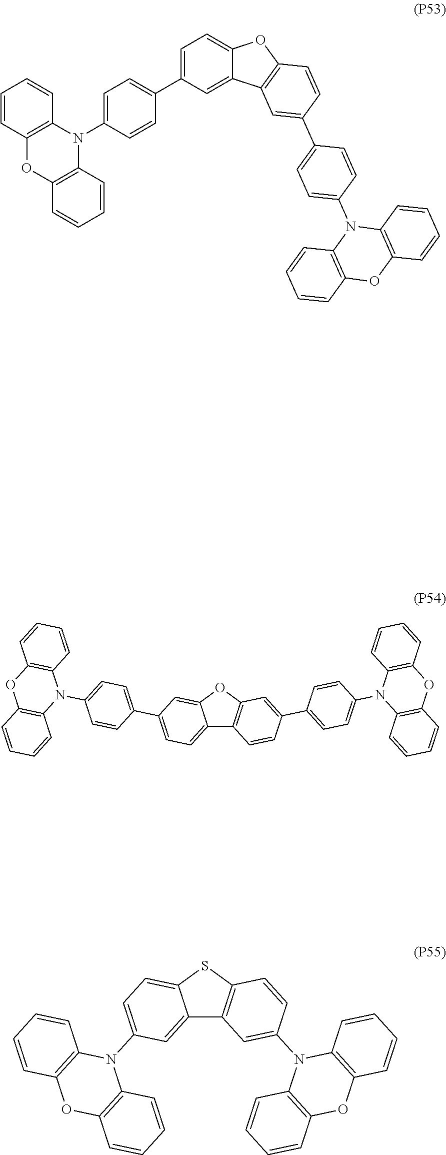

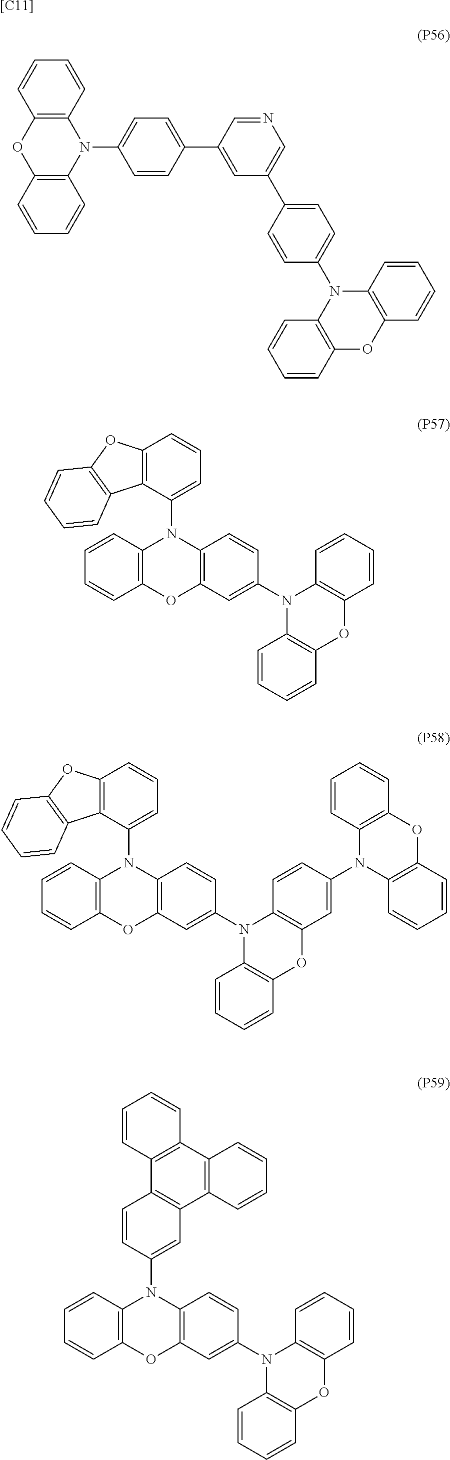

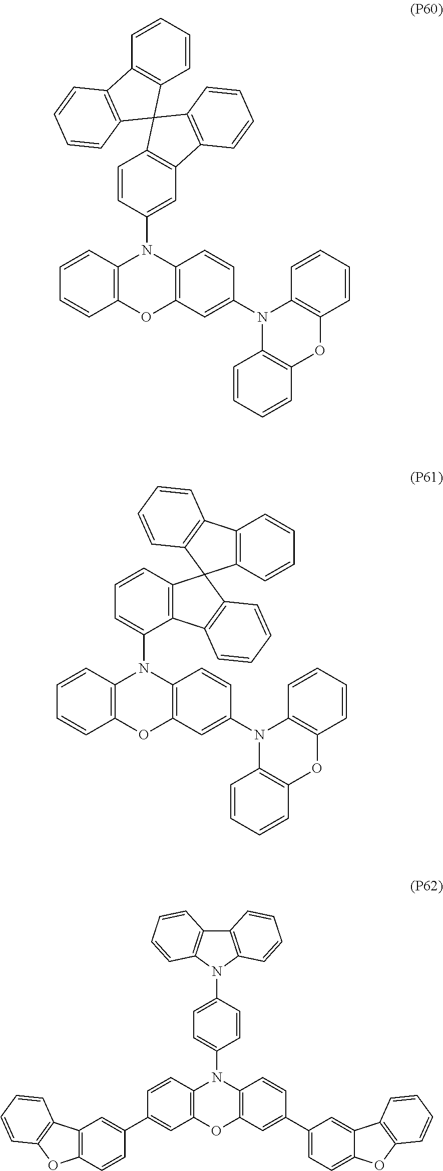

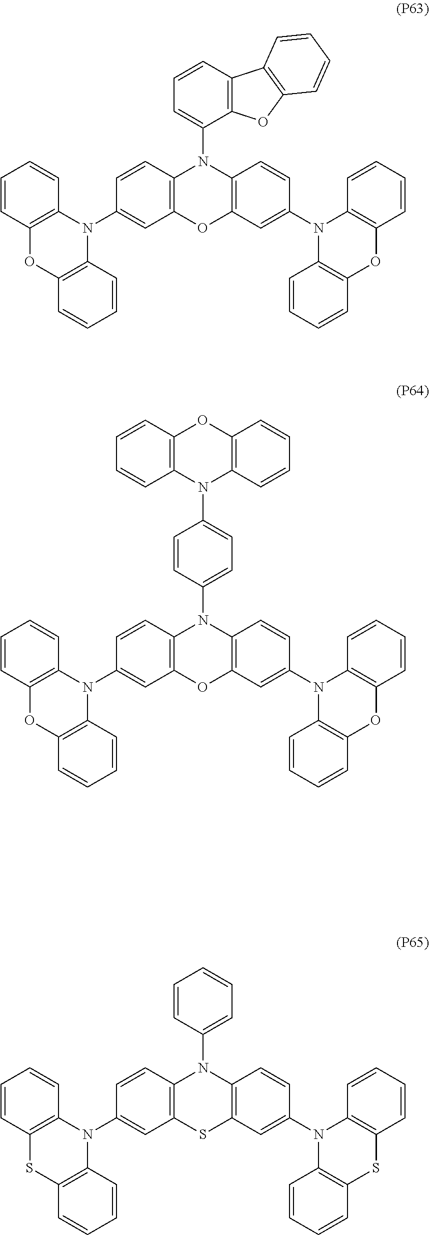

The material for a photoelectric conversion device of the present invention can be obtained by: synthesis by methods of various organic synthetic reactions established in the field of the organic synthetic chemistry including coupling reactions such as Suzuki coupling, Stille coupling, Grignard coupling, Ullmann coupling, Buchwald-Hartwig reaction, and Heck reaction, using commercially available reagents as raw materials; and then purification by using a known method such as recrystallization, column chromatography, and sublimation and purification. The method is not limited to this method.

The material for a photoelectric conversion device of the present invention preferably has an energy level of highest occupied molecular orbital (HOMO) obtained by structural optimization calculation with a density functional calculation B3LYP/6-31G (D) of −4.0 eV or lower, more preferably within a range of −5.0 eV to −4.5 eV.

An energy level of lowest unoccupied molecular orbital (LUMO) obtained by the above structural optimization calculation is preferably −2.5 eV or higher, more preferably within a range of −2.5 eV to −0.9 eV.

In the material for a photoelectric conversion device of the present invention, a difference (absolute value) between the HOMO energy level and the LUMO energy level is preferably within a range of 2.0 to 5.0 eV, and more preferably within a range of 2.5 to 4.0 eV.

The material for a photoelectric conversion device of the present invention preferably has a hole mobility of 1×10⁻⁶ cm²/Vs to 1 cm²/Vs, more preferably has a hole mobility of 1×10⁻⁵ cm²/Vs to 1×10⁻¹ cm²/Vs. The hole mobility can be evaluated by known methods such as a method with a FET-type transistor device, a method with a time-of-flight method, and an SCLC method.

The material for a photoelectric conversion device of the present invention is preferably amorphous. The amorphousness can be confirmed by various methods, and can be confirmed by, for example, detecting no peak in an XRD method or by detecting no endothermic peak in a DSC method.

Next, a photoelectric conversion device for imaging using the material for a photoelectric conversion device of the present invention will be described, but a structure of the photoelectric conversion device for imaging of the present invention is not limited thereto. The description will be made with reference to the drawing.

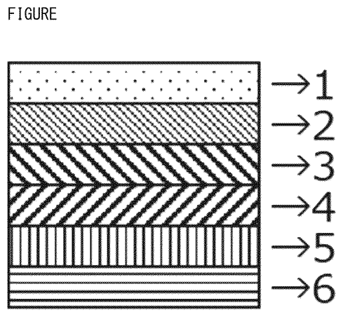

FIG. 1 is a sectional view schematically illustrating a structure of the photoelectric conversion device for imaging using the material for the photoelectric conversion device for imaging of the present invention. In FIG. 1, 1 represents a substrate, 2 represents an electrode, 3 represents an electron blocking layer, 4 represents a photoelectric conversion layer, 5 represents a hole blocking layer, and 6 represents an electrode. The photoelectric conversion device is not limited to the structure in FIG. 1, and adding or omitting a layer can be made as necessary. It is also possible to provide an inverse structure to that in FIG. 1, namely, stack the electrode 6, the hole blocking layer 5, the photoelectric conversion layer 4, the electron blocking layer 3, and the electrode 2 in the listed order on the substrate 1, and in this case, a layer can be added or omitted, if needed. Herein, layers other than the electrodes like an anode and a cathode, as described above, which are located on the substrate and constitute a stacked structure in the photoelectric conversion device for imaging, may also be collectively referred to as an organic layer.

Hereinafter, each member and each layer of the photoelectric conversion device of the present invention will be described.

### —Substrate—

The photoelectric conversion device is preferably supported on a substrate. This substrate is not particularly limited, and a substrate composed of glass, a transparent plastic, quartz, or the like may be used, for example.

### —Electrode—

An electrode used for the photoelectric conversion device for imaging of the present invention has a function of trapping a hole and an electron generated in the photoelectric conversion layer. A function to let light enter the photoelectric conversion layer is also required. Thus, at least one of two electrodes is desirably transparent or semi-transparent. A material used for the electrode is not particularly limited as long as it has conductivity, and examples thereof include: conductive transparent materials, such as ITO, IZO, SnO₂, ATO (antimony-doped tin oxide), ZnO, AZO (Al-doped zinc oxide), GZO (gallium-doped zinc oxide), TiO₂, and FTO; metals, such as gold, silver, platinum, chromium, aluminum, iron, cobalt, nickel, and tungsten; inorganic conductive substances, such as copper iodide and copper sulfide; and conductive polymers, such as polythiophene, polypyrrole, and polyaniline. A plurality of these materials may be mixed to use as necessary. In addition, two or more layers thereof may be stacked.

### —Photoelectric Conversion Layer—

The photoelectric conversion layer is a layer in which a hole and an electrode are generated by charge separation of an exciton generated by the incident light. The photoelectric conversion layer may be formed with a single photoelectric converting material, or may be formed by combination with a P-type organic semiconductor material being a hole transport material and an N-type organic semiconductor material being an electron transport material. Two or more kinds of the P-type organic semiconductor may be used, and two or more kinds of the N-type organic semiconductor may be used. One or more kinds of these P-type organic semiconductor and/or N-type organic semiconductor desirably use a dye material having a function of absorbing light with a desired wavelength in the visible region. As the P-type organic semiconductor material being the hole transport material, the material for a photoelectric conversion device of the present invention can be used.

The P-type organic semiconductor material may be any material having a hole transportability. The material for a photoelectric conversion device of the present invention is preferably used, but another P-type organic semiconductor material may be used. Two or more types of the compound represented by the general formula (1) (the material for a photoelectric conversion device of the present invention) may be mixed to use. Furthermore, the above compound and another P-type organic semiconductor material may be mixed to use.

The another P-type organic semiconductor material may be any material having the hole transportability, and examples thereof include: aromatic compounds such as a naphthalene derivative, an anthracene derivative, a phenanthrene derivative, a pyrene derivative, a chrysene derivative, a naphthacene derivative, a triphenylene derivative, a perylene derivative, a fluoranthene derivative, a fluorene derivative, a cyclopentadiene derivative, a furan derivative, a thiophene derivative, a pyrrole derivative, a benzofuran derivative, a dibenzothiophene derivative, a dinaphthothienothiophene derivative, an indole derivative, a pyrazoline derivative, a dibenzofuran derivative, a dibenzothiophene derivative, a carbazole derivative, and indolocarbazole; an aromatic amine derivative, a styrylamine derivative, a benzidine derivative, a porphyrin derivative, a phthalocyanine derivative, and a quinacridone derivative.

As the P-type organic semiconductor material, a polymer material may be used. Examples of the polymer P-type organic semiconductor material include a polyphenylene-vinylene derivative, a polyparaphenylene derivative, a polyfluorene derivative, a polyvinylcarbazole derivative, and a polythiophene derivative. Two or more kinds selected from the compounds represented by (1) of the present invention, the P-type organic semiconductor material, and the polymer P-type organic semiconductor material may be mixed to use.

The N-type organic semiconductor material may be any material having the electron transportability, and examples thereof include naphthalenetetracarboxylic diimide and perylenetetracarboxylic diimide, fullerenes (fullerene derivative), and azole derivatives such as imidazole, thiazole, thiadiazole, oxazole, oxadiazole, and triazole. Two or more kinds of materials selected from the N-type organic semiconductor materials may be mixed to use.

### —Electron Blocking Layer—

The electron blocking layer is provided in order to inhibit a dark current generated by injecting an electron from one electrode into the photoelectric conversion layer when a bias voltage is applied between the two electrodes. The electron blocking layer also has a function of hole transportation for transporting a hole generated by charge separation in the photoelectric conversion layer toward the electrode. A single layer or multiple layers of the electron blocking layer can be disposed as necessary. For the electron blocking layer, a P-type organic semiconductor material being the hole transport material can be used. The P-type organic semiconductor material may be any material having the hole transportability. Although the compound represented by the general formula (1) is preferably used, another P-type organic semiconductor material may be used. In addition, the compound represented by the general formula (1) and the other P-type organic semiconductor material as noted above or a polymer P-type organic semiconductor material may be mixed to use.

### —Hole Blocking Layer—

The hole blocking layer is provided in order to inhibit a dark current generated by injecting a hole from one electrode into the photoelectric conversion layer when a bias voltage is applied between the two electrodes. The hole blocking layer also has a function of electron transportation for transporting an electron generated by charge separation in the photoelectric conversion layer toward the electrode. A single layer or multiple layers of the hole blocking layer can be disposed as necessary. For the hole blocking layer, the N-type organic semiconductor material having the electron transportability can be used. The N-type organic semiconductor material may be any material having the electron transportability, and examples thereof include: polycyclic aromatic multivalent carboxylic anhydride or imidized products thereof, such as naphthalenetetracarboxylic diimide and perylenetetracarboxylic diimide; fullerenes (fullerene derivative), such as C60 and C70; azole derivatives, such as imidazole, thiazole, thiadiazole, oxazole, oxadiazole, and triazole; a tris(8-quinolinolate)aluminum (III) derivative, a phosphine oxide derivative, a nitro-substituted fluorene derivative, a diphenylquinone derivative, a thiopyran dioxide derivative, a carbodiimide, a fluorenylidene methane derivative, an anthraquinodimethane derivative and an anthrone derivative, a bipyridine derivative, a quinoline derivative, and an indolocarbazole derivative. Two or more kinds of materials selected from the N-type organic semiconductor materials may be mixed to use.

Hydrogen in the material of the present invention may be deuterium. That is, alkyl group, or a part or all of hydrogens on the aromatic ring, in addition to hydrogens on the aromatic ring in the general formula (1), (2a), (2b), (2c), (2d), (2e), (3a), (3b), (3c), (3d), (3e), (36), (3g), (3h), (31), (31), (3k), and (3m) and including the substituent such as Ar¹, AR², Ar³, AR⁴, R¹ and R², may be a deuterium.

Furthermore, a part or all of hydrogens in a compound used as the N-type organic semiconductor material and the P-type organic semiconductor material may be deuterium.

A method for producing a film of each layer in producing the photoelectric conversion device for imaging of the present invention is not particularly limited. The photoelectric conversion device may be produced by any one of dry process and wet process.

The organic layer containing the material for a photoelectric conversion device of the present invention may be a plurality of layers as necessary.

### EXAMPLES

Hereinafter, the present invention will be described in more detail with Examples, but the present invention is not limited to these Examples,

### Calculation Example

### Calculation of HOMO and LUMO Values

Calculated were HOMO and LUMO for the compounds P1, P4, P20, P38, P39, P41, P50, P51, P52, P55, P63, P64 and P65. The calculation was performed by using a density functional theory (DFT), using Gaussian as a calculation program, and with structural optimization calculation of a density functional calculation B3LYP/6-31G (d). Table 1 shows the results. It can be mentioned that any of the materials of the present invention has preferable HOMO and LUMO values.

As comparison, HOMO and LUMO for the compounds H1, H2, H3, H4 and H5 were calculated by the same method as the above method. Table 1 shows the results.

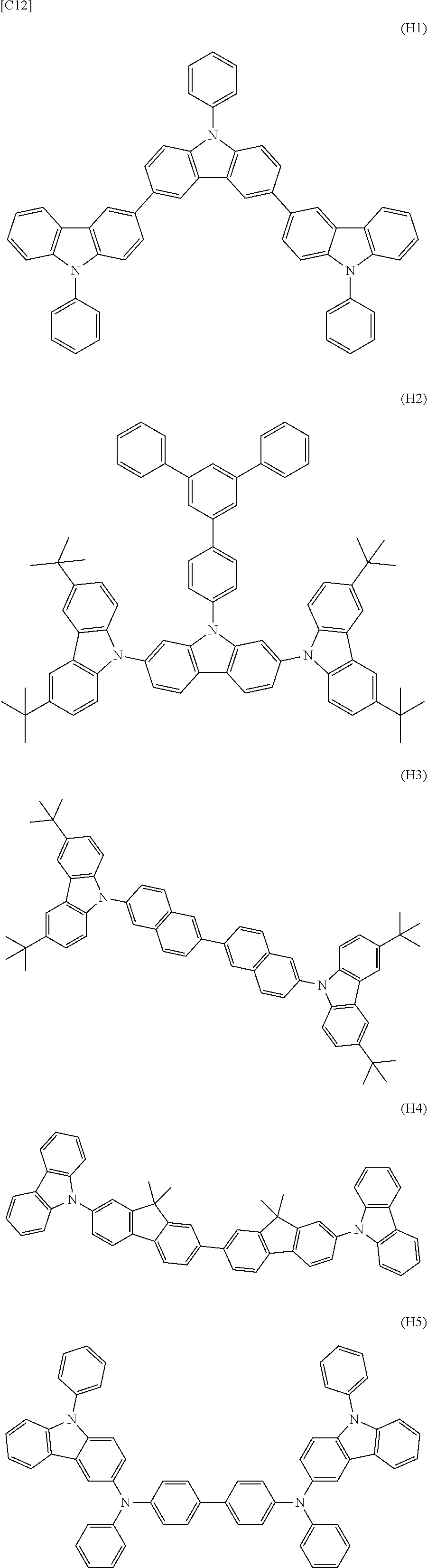

TABLE 1

| Com- | HOMO | LUMO |
|---|---|---|
| pound | [eV] | [eV] |
| P1 | −4.9 | −0.9 |
| P4 | −4.6 | −0.9 |
| P20 | −4.8 | −1.6 |
| P38 | −4.6 | −1.4 |
| P39 | −4.7 | −1.2 |
| P41 | −4.6 | −1.3 |
| P50 | −4.6 | −1.6 |
| P51 | −4.7 | −1.5 |
| P52 | −4.7 | −1.5 |
| P55 | −4.7 | −1.5 |
| P63 | −4.6 | −1.5 |
| P64 | −4.7 | −1.3 |
| P65 | −4.9 | −1.1 |
| H1 | −4.9 | −0.7 |
| H2 | −5.0 | −1.2 |
| H3 | −4.9 | −1.3 |
| H4 | −5.2 | −1.5 |
| H5 | −4.5 | −0.8 |

Synthesis examples of the compounds P1, P39, and P51 will be described below as representative examples. The other compounds were also synthesized in the similar method.

### Synthesis Example 1 (Synthesis of Compound P1)

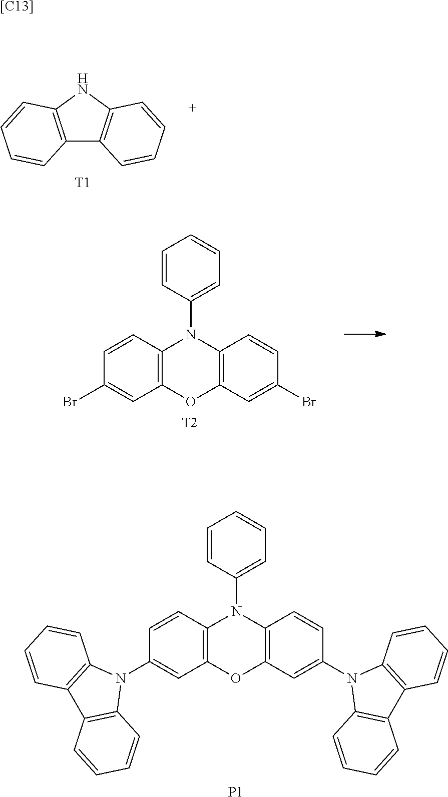

Into a three-necked 300-ml flask with degassed and nitrogen-replenished, T1 (16.0 mmol), T2 (6.9 mmol), bis(tri-tert-butylphosphine) palladium (0) (0, 6 mmol), and sodium tert-butoxide (21.1 mmol) were added, and 40 ml of xylene was added thereinto, and then the mixture was stirred at 150° C. for 6 hours. The mixture was once cooled to a room temperature, then the reaction liquid was concentrated to dryness, purified by column chromatography, and then reprecipitated with xylene and isopropanol to obtain the compound 1 (white solid). The yield was 678. The obtained solid was evaluated by the XRD method, but no peak was detected. It was thus confirmed that the compound P1 was amorphous. APCI-TOFMS m/z 590 [M+1]⁺

### Synthesis Example 2 (Synthesis of Compound P39)

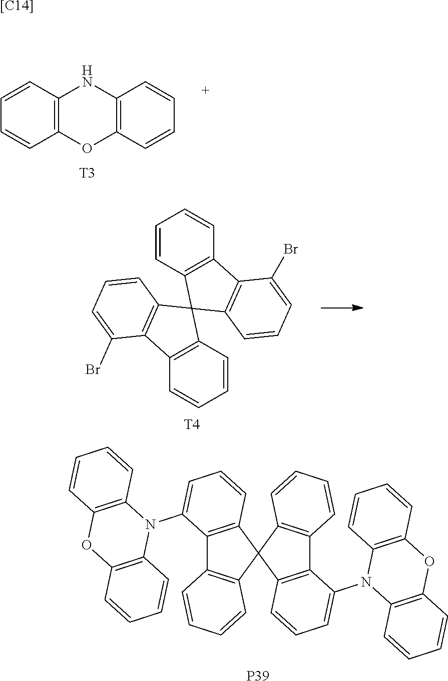

The compound P39 (white solid) was obtained in the same manner as in Synthesis Example 1 except that T1 was changed to T3 and T2 was changed to T4. The yield was 21%. The obtained solid was evaluated by the XRD method, but no peak was detected. APCI-TOFMS m/z 679 [M+1]⁺

### Synthesis Example 3 (Synthesis of Compound P51)

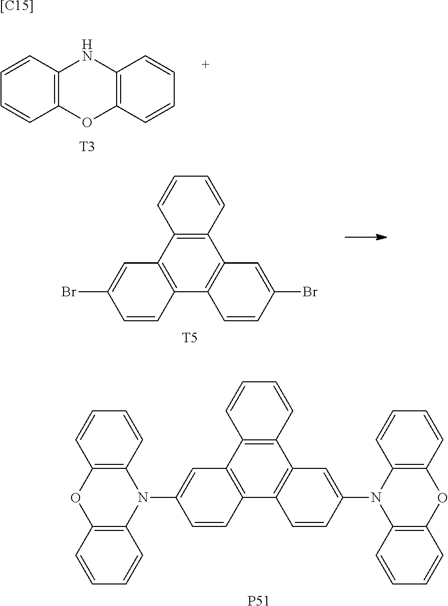

The compound P51 (white solid) was obtained in the same manner as in Synthesis Example 1 except that T3 was changed to T5. The yield was 438. The obtained solid was evaluated by the XRD method, but no peak was detected. APCI-TOFMS m/z 591 [M+1]⁺

### Measurement of Charge Mobility

On an electrode composed of ITO with 110 nm in film thickness formed on a glass substrate, the compound 1 was produced to a film as an organic layer by a vacuum deposition method under a condition that a film thickness was approximately 3 μm. Subsequently, charge mobility was measured by a time-of-flight method using a device in which aluminum (Al) was formed with 70 nm in thickness as an electrode. The hole mobility was 1.3×10⁻⁴ cm²/Vs.

The hole mobility of each compound was measured by the same method as above except that compounds shown in Table 2 were used instead of the compound 1.

Table 2 shows the results,

TABLE 2

| Com- | Hole mobility |
|---|---|
| pound | [cm²/Vs] |
| P1 | 1.3 × 10⁻⁴ |
| P4 | 2.2 × 10⁻⁴ |
| P38 | 2.6 × 10⁻⁵ |
| P39 | 7.1 × 10⁻⁴ |
| P41 | 1.2 × 10⁻⁵ |
| P51 | 2.4 × 10⁻⁴ |
| P55 | 4.5 × 10⁻⁴ |
| P65 | 2.5 × 10⁻⁴ |
| H1 | 1.2 × 10⁻⁵ |
| H2 | 9.3 × 10⁻⁶ |
| H3 | 1.1 × 10⁻⁵ |
| H4 | 6.5 × 10⁻⁴ |
| H5 | 2.9 × 10⁻³ |

### Example 1

On an electrode composed of ITO with 70 nm in film thickness formed on a glass substrate, a 10-nm film of the compound P1 was formed with a vacuum degree of 4.0×10⁻⁵ Pa as an electron blocking layer. Then, 2Ph-BTBT (material having the hole transportability), F6-SubPc-OC6F5 (dye material), and fullerene (C60, material having the electron transportability) were co-deposited at a deposition rate ratio of 4:4:2 with 200 nm to form a film as a photoelectric conversion layer. Subsequently, 10-nm of dpy-NDI was deposited to form a hole blocking layer. Finally, an aluminum film was formed with 70 nm in thickness as an electrode to produce a photoelectric conversion device. A current in a dark place (dark current) was 3.7×10⁻¹⁰ A/cm² with the electrodes of ITO and aluminum and with applying a voltage of 2.6 V. When a voltage of 2.6 V was applied and the ITO electrode side was irradiated with light with an LED adjusted to be an irradiation light wavelength of 500 nm and 1.6 μW from a height of 10 cm, a current (bright current) was 3.0×10⁻⁷ A/cm². A contrast ratio was 8.1×10² with applying a voltage of 2.6 V. Table 3 shows the results.

### Examples 2 to 13

Photoelectric conversion devices were produced in the same manner as in Example 1 except that compounds shown in Table 3 were used as the electron blocking layer.

### Comparative Examples 1 to 5

Photoelectric conversion devices were produced in the same manner as in Example 1 except that compounds shown in Table 3 were used as the electron blocking layer.

Table 3 shows the results in Examples 1 to 13 and Comparative Examples 1 to 5.

Table 4 shows the calculation results of HOMO and LUMO of the compounds used in Examples and Comparative Examples, other than the compounds shown in Table 1 above. The calculation was performed as in the above case, by using a density functional theory (DFT), using Gaussian as a calculation program, and with structural optimization calculation of a density functional calculation B3LYP/6-31G (d).

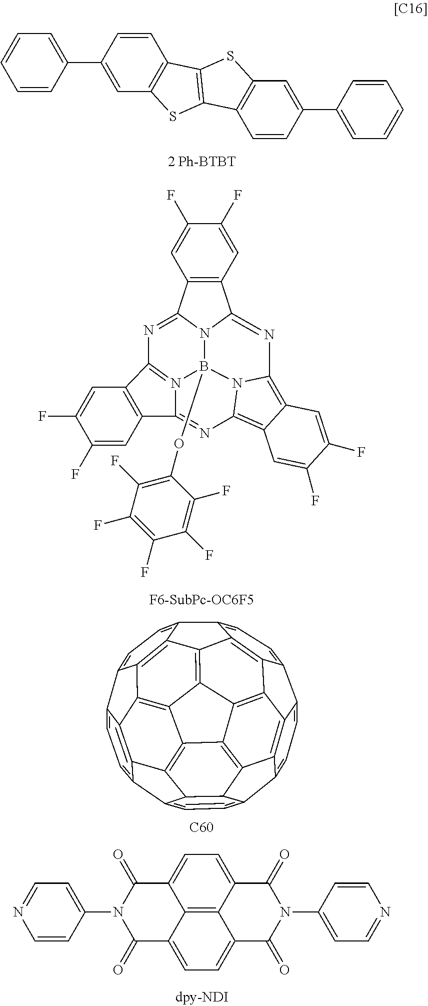

TABLE 3

| | | | | | Difference in |
|---|---|---|---|---|---|
| | Compound | Current value | Current value with | | energy level |
| | in electron | in dark place | light irradiation | Contrast | \|h1−e1\| |
| | blocking layer | [A/cm²] | [A/cm²] | ratio | [eV] |
| Example 1 | P1 | 3.7 × 10⁻¹⁰ | 3.0 × 10⁻⁷ | 8.1 × 10² | 0.5 |
| Example 2 | P4 | 2.3 × 10⁻¹⁰ | 2.6 × 10⁻⁷ | 1.1 × 10³ | 0.8 |
| Example 3 | P20 | 4.5 × 10⁻¹⁰ | 2.8 × 10⁻⁷ | 6.2 × 10² | 0.6 |
| Example 4 | P38 | 4.4 × 10⁻¹⁰ | 2.7 × 10⁻⁷ | 6.1 × 10² | 0.8 |
| Example 5 | P39 | 3.7 × 10⁻¹⁰ | 3.0 × 10⁻⁷ | 8.1 × 10² | 0.7 |
| Example 6 | P41 | 4.4 × 10⁻¹⁰ | 2.6 × 10⁻⁷ | 5.9 × 10² | 0.8 |
| Example 7 | P50 | 3.9 × 10⁻¹⁰ | 3.0 × 10⁻⁷ | 7.7 × 10² | 0.8 |
| Example 8 | P51 | 3.5 × 10⁻¹⁰ | 2.9 × 10⁻⁷ | 8.3 × 10² | 0.7 |
| Example 9 | P 52 | 3.4 × 10⁻¹⁰ | 2.8 × 10⁻⁷ | 8.2 × 10² | 0.7 |
| Example 10 | P55 | 3.4 × 10⁻¹⁰ | 2.8 × 10⁻⁷ | 8.2 × 10² | 0.7 |
| Example 11 | P63 | 3.6 × 10⁻¹⁰ | 2.7 × 10⁻⁷ | 7.5 × 10² | 0.8 |
| Example 12 | P64 | 3.7 × 10⁻¹⁰ | 2.9 × 10⁻⁷ | 7.8 × 10² | 0.7 |
| Example 13 | P65 | 1.9 × 10⁻¹⁰ | 2.7 × 10⁻⁷ | 1.4 × 10³ | 0.5 |
| Comparative | H1 | 7.7 × 10⁻¹⁰ | 2.4 × 10⁻⁷ | 3.1 × 10² | 0.5 |
| Example 1 | | | | | |
| Comparative | H2 | 6.1 × 10⁻¹⁰ | 2.0 × 10⁻⁷ | 3.3 × 10² | 0.4 |
| Example 2 | | | | | |
| Comparative | H3 | 3.9 × 10⁻¹⁰ | 2.0 × 10⁻⁷ | 5.1 × 10² | 0.5 |
| Example 3 | | | | | |
| Comparative | H4 | 5.2 × 10⁻¹⁰ | 2.2 × 10⁻⁷ | 4.2 × 10² | 0.2 |
| Example 4 | | | | | |
| Comparative | H5 | 1.8 × 10⁻⁹ | 1.9 × 10⁻⁷ | 1.1 × 10² | 0.9 |
| Example 5 | | | | | |

TABLE 4

| | HOMO | LUMO |
|---|---|---|
| Compound | [eV] | [eV] |
| 2Ph- BTBT | −5.4 | −1.5 |
| F6-SubPc-OC6F5 | −5.8 | −3.0 |
| C60 | −6.0 | −3.2 |
| dpy-NDI | ⁻7.5 | −4.5 |

It is found from the results in Table 3 that the photoelectric conversion devices using the compound of the present invention exhibit a low dark current value and a high contrast ratio.

### INDUSTRIAL APPLICABILITY

The material for a photoelectric conversion device for imaging of the present invention can achieve appropriate move of the hole and the electron in the photoelectric conversion device, and consequently enables to reduce a leakage current generated by applying a bias voltage during the conversion of light into electric energy. As a result, a photoelectric conversion device that achieves a low dark current value and a high contrast ratio can be obtained. The material of the present invention is useful as a material for a photoelectric conversion device for a photoelectric-converting film-stacked imaging device.

### REFERENCE SIGNS LIST

1 Substrate, 2 Electrode, 3 Electron blocking layer, 4 Photoelectric conversion layer, 5 Hole blocking layer, 6 Electrode.

1. A photoelectric conversion device comprising one or two or more organic layers between two electrodes, wherein at least one of the organic layers contains a material for a photoelectric conversion device represented by the following general formula (1): 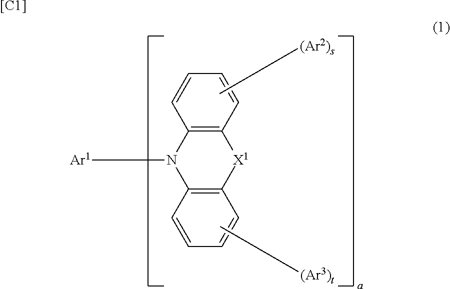
wherein X¹ each independently represents O, S, or N—R¹;
R¹ each independently represents an alkyl group having 1 to 20 carbon atoms, a substituted or unsubstituted aromatic hydrocarbon group having 6 to 30 carbon atoms, or a substituted or unsubstituted heteroaromatic group having 3 to 17 carbon atoms;
Ar¹ to Ar³ each independently represent deuterium, an alkyl group having 1 to 20 carbon atoms, a substituted or unsubstituted aromatic hydrocarbon group having 6 to 30 carbon atoms, a substituted or unsubstituted heteroaromatic group having 3 to 17 carbon atoms, or a group having a tricyclic condensed-ring structure represented by any of the following formulae (2a) to (2e), or a substituted or unsubstituted linked aromatic group in which two to five of any of a substituted or unsubstituted aromatic hydrocarbon group having 6 to 30 carbon atoms, a substituted or unsubstituted heteroaromatic group having 3 to 17 carbon atoms, and the following formulae (2a) to (2e) are linked, wherein Ar¹ to Ar³ are each independently represented by the following formula (2c) or (2d) when the tricyclic condensed-ring structure is contained at the terminal, or Ar¹ to Ar³ are each independently represented by the formula (2a), (2b), or (2e) when the tricyclic condensed-ring structure is not contained at the terminal and there are two bonding sites; 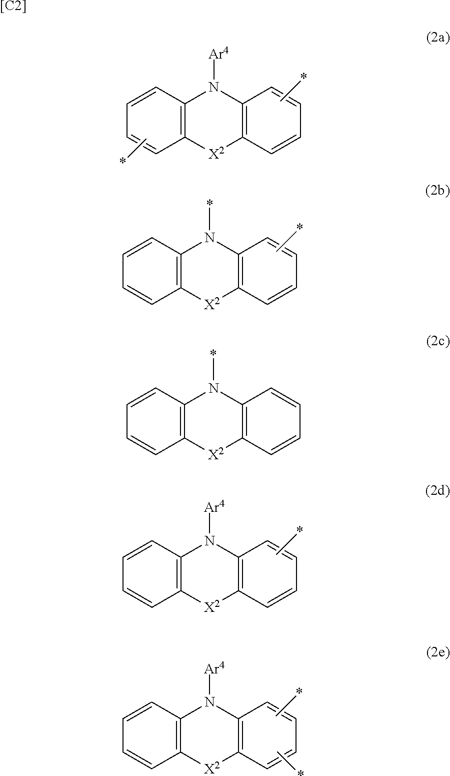
“*” represents a point of bonding;
Ar⁴ independently represents hydrogen, deuterium, an alkyl group having 1 to 20 carbon atoms, a substituted or unsubstituted aromatic hydrocarbon group having 6 to 30 carbon atoms, or a substituted or unsubstituted linked aromatic group in which two to five of these aromatic groups are linked;
X² each independently represents a single bond, O, S, or N—R², and R² each independently represents an alkyl group having 1 to 20 carbon atoms, a substituted or unsubstituted aromatic hydrocarbon group having 6 to 30 carbon atoms, or a substituted or unsubstituted heteroaromatic group having 3 to 17 carbon atoms;
“s” and “t” independently represent the number of substitution, “s” and “t” independently represent an integer of 0 to 2, and “a” represents the number of substitution and represents an integer of 1 or 2;
provided that, when “a” is 1, at least one of Ar¹ to Ar³ contains the group having the tricyclic condensed-ring structure represented by any of the formulae (2a) to (2e).

2. The photoelectric conversion device according to claim 1, wherein in the general formula (1), Ar¹ contains any ring structure represented by the following general formulae (3a) to (3m):

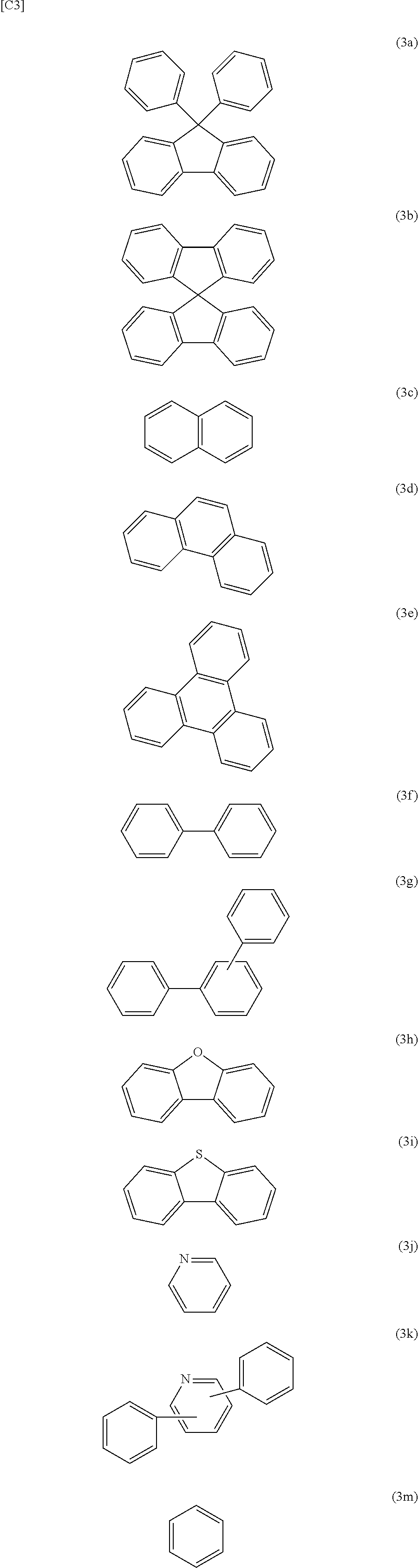

3. The photoelectric conversion device according to claim 1, wherein in the general formula (1), X² is represented by a single bond, O, or S.

4. The photoelectric conversion device according to claim 1, wherein in the general formula (1), Ar¹ contains at least one substituted or unsubstituted heteroaromatic group having 3 to 17 carbon atoms and containing an oxygen or sulfur atom.

5. The photoelectric conversion device according to claim 2, wherein in the general formula (1), Ar¹ is any ring structure represented by the general formulae (3a) to (3m).

6. The photoelectric conversion device according to claim 1, wherein in the general formula (1), “a” is 1 and at least two of Ar¹ to Ar³ contain the group having the tricyclic condensed-ring structure represented by any of the formulae (2a) to (2e).

7. The photoelectric conversion device according to claim 1, wherein in the general formula (1), “a” is 1 and at least one of Ar¹ to Ar³ contains the group having the tricyclic condensed-ring structure represented by the formula (2c).

8. The photoelectric conversion device according to claim 7, wherein in the general formula (1), at least two of Ar¹ to Ar³ contain the group having the tricyclic condensed-ring structure represented by the formula (2c).

9. The photoelectric conversion device according to claim 1, wherein, in the material for a photoelectric conversion device represented by the general formula (1), an energy level of highest occupied molecular orbital (HOMO) obtained by structural optimization calculation with a density functional calculation B3LYP/6-31G (d) is −4.0 eV or less.

10. The photoelectric conversion device according to claim 1, wherein, in the material for a photoelectric conversion device represented by the general formula (1), an energy level of lowest unoccupied molecular orbital (LUMO) obtained by structural optimization calculation with a density functional calculation B3LYP/6-31G (d) is −2.5 eV or more.

11. The photoelectric conversion device according to claim 1, wherein the material for a photoelectric conversion device represented by the general formula (1) has a hole mobility of 1×10⁻⁶ cm²/Vs or more.

12. The photoelectric conversion device according to claim 1, wherein the material for a photoelectric conversion device represented by the general formula (1) is amorphous.

13. The photoelectric conversion device according to claim 1, wherein the material for a photoelectric conversion device represented by the general formula (1) is used as a bole transport material.

14. The photoelectric conversion device according to claim 1, wherein the photoelectric conversion device is a photoelectric conversion device for imaging, comprising a photoelectric conversion layer and an electron blocking layer as organic layers between two electrodes, and the electron blocking layer contains the material for a photoelectric conversion device represented by the general formula (1).

15. The photoelectric conversion device according to claim 14, wherein an absolute value of a difference in energy level of HOMO between the material for a photoelectric conversion device represented by the general formula (1) and the hole transport material contained in the photoelectric conversion layer is 1.0 eV or less.

16. The photoelectric conversion device according to claim 14, wherein the photoelectric conversion layer contains an electron transport material.

17. The photoelectric conversion device according to claim 16, wherein the photoelectric conversion layer contains a fullerene derivative as the electron transport material.

18. The photoelectric conversion device according to claim 16, wherein the photoelectric conversion layer contains a material having a backbone containing at least two thiophene rings, as the hole transport material.
